SEQUENCE LISTING (4 sequences)

SEQ ID NO: 1 - RNA, 5589 nt, Homo sapiens
  source 1..5589: mol_type = mRNA
ggcgggggaggcgccgggaagtcgacggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaaaattacaggaaaagatatccatgacacagccagcagtgtggcacgtgggctacaattccagcgtggccttcagttctgcacacgtgcgtcaaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 2 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 3 - RNA, 5892 nt, Homo sapiens
  source 1..5892: mol_type = mRNA
gcgtcttccggcgcccgcggaggaggcgagggtgggacgctgggcggagcccgagtttaggaagaggaggggacggctgtcatcaatgaagtcatattcataatctagtcctctctccctctgtttctgtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagataggtgtaacgtggagaaggtgcccagtaattctcagttggaaatagaaggaaacagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 4 - protein, 1590 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

(19) United States
(12) Patent Application Publication
(10) Pub. No.: US 2026/0144797 A1
(43) Pub. Date: May 28, 2026

(54) METHODS OF REDUCING OR PREVENTING METASTASES

(71) Applicant: FOGHORN THERAPEUTICS INC., Cambridge, MA (US)

(72) Inventors: Jessica Y. WAN, Newton, MA (US); Jessica C. PIEL, Natick, MA (US); Martin F. HENTEMANN, Carlisle, MA (US)

(21) Appl. No.: 19/121,614

(22) PCT Filed: Oct. 17, 2023
(86) PCT No.: PCT/US2023/077088

§ 371 Date: Apr. 16, 2025

Related U.S. Application Data
(60) Provisional application No. 63/416,835, filed on Oct. 17, 2022.

(51) Int. Cl. A61K 31/5377; A61K 9/00; A61K 9/48; A61K 45/06; A61P 35/00

(52) U.S. Cl. CPC A61K 31/5377; A61K 9/0053; A61K 9/4816; A61K 9/4858; A61K 9/4866; A61K 45/06; A61P 35/00

(57) ABSTRACT

The present disclosure features methods useful for the treatment of BAF complex-related cancers.

### SEQUENCE LISTING

The instant application contains a Sequence Listing which has been submitted electronically in XML format and is hereby incorporated by reference in its entirety. Said XML copy, created on Oct. 17, 2023, is named “51121-085WO2_Sequence_Listing_10_17_23” and is 19,006 bytes in size.

### BACKGROUND

The present disclosure relates to compounds and methods useful for modulating BRG1- or BRM-associated factors (BAF) complexes. In particular, the present disclosure relates to compounds and methods useful for treatment of disorders associated with BAF complex function, such as cancer.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present disclosure features methods of treating metastatic cancer using an agent that reduces the level and/or activity of BRG1 and/or BRM.

In an aspect, the disclosure provides a method of slowing the spread of a migrating cancer in a subject having a high risk of the spread of the migrating cancer, the method including administering to the subject an effective amount of an agent that reduces the level and/or activity of BRG1 and/or BRM.

In an aspect, the disclosure provides a method of reducing the rate of tumor seeding of a cancer in a subject having a high risk of tumor seeding of a cancer, the method including administering to the subject an effective amount of an agent that reduces the level and/or activity of BRG1 and/or BRM.

In an aspect, the disclosure provides a method of reducing or treating metastatic nodule-forming of a cancer in a subject having a high risk of metastatic nodule-forming of a cancer, the method including administering to the subject an effective amount of an agent that reduces the level and/or activity of BRG1 and/or BRM.

In an aspect, the disclosure provides a method of reducing metastatic risk of a cancer in a subject having a high metastatic risk, the method including administering to the subject an effective amount of an agent that reduces the level and/or activity of BRG1 and/or BRM.

In an aspect, the disclosure provides a method for treating metastatic cancer in a subject having a high metastatic risk, the method including administering to the subject an effective amount of an agent that reduces the level and/or activity of BRG1 and/or BRM.

In an aspect, the disclosure provides a method for inhibiting cancer cell invasion or migration in a subject having a high risk of cancer cell invasion or migration, the method including administering to the subject an effective amount of an agent that reduces the level and/or activity of BRG1 and/or BRM.

In some embodiments, the cancer is melanoma, non-small cell lung cancer, small-cell lung cancer, ovarian cancer, breast cancer, esophagogastric cancer, oral cancer, glioblastoma, colon cancer, hematologic cancer, sarcoma, or neuroblastoma.

In some embodiments, the cancer is melanoma.

In some embodiments, the melanoma is uveal melanoma, mucosal melanoma, or cutaneous melanoma.

In some embodiments, the melanoma is uveal melanoma.

In some embodiments, the cancer is a hematologic cancer.

In some embodiments, the hematologic cancer is acute myeloid leukemia.

In some embodiments, the acute myeloid leukemia is refractory acute myeloid leukemia.

In some embodiments, the acute myeloid leukemia is relapsed acute myeloid leukemia.

In some embodiments, the cancer is breast cancer.

In some embodiments, the breast cancer is an ER positive breast cancer, an ER negative breast cancer, triple positive breast cancer, or triple negative breast cancer.

In some embodiments, the breast cancer is a triple negative breast cancer.

In some embodiments, the cancer is an oral cancer that is an oral squamous cancer.

In an aspect, the disclosure provides a method of treating GEP2 uveal melanoma in a subject in need thereof, and the method including administering to the subject an agent that reduces the level and/or activity of BRG1 and/or BRM.

In an aspect, the disclosure provides a method of transforming a GEP2 uveal melanoma to a GEP1 uveal melanoma in a subject in need thereof, the method including administering to the subject an agent that reduces the level and/or activity of BRG1 and/or BRM.

In some embodiments, the cancer expresses BRG1 and/or BRM protein.

In some embodiments, the cancer expresses PRAME. In some embodiments, the cancer expresses PROS1.

In some embodiments, the cancer underexpresses BAP1, MGP, CXCL14, CLCA2, S100A8, BTG1, SAP130, ARG1, KRT6B, GJA, ID2, EIF1B, S100A9, CRABP2, KRT14, ROBO1, RBM23, TACSTD2, DSC1, SPRR1B, TRIM29, AQP3, TYRP1, PPL, LTA4H, or CST6. In some embodiments, the subject underexpresses BAP1, MGP, CXCL14, CLCA2, S100A8, BTG1, SAP130, ARG1, KRT6B, GJA, ID2, EIF1B, S100A9, CRABP2, KRT14, ROBO1, RBM23, TACSTD2, DSC1, SPRR1B, TRIM29, AQP3, TYRP1, PPL, LTA4H, or CST6. In some embodiments, the subject overexpresses SPP1. In some embodiments, the cancer overexpresses SPP1.

In some embodiments, the subject expresses PRAME. In some embodiments, the subject expresses PROS1.

In some embodiments, the subject overexpresses SNAI1, TCF4, TWIST1, FOXC2, IL1RN, MMP2, SOX10, WNT11, MMP3, PDGFRB, or JAG1. In some embodiments, the subject has a breast cancer (e.g., a triple negative breast cancer) and overexpresses JAG1. In some embodiments, the cancer overexpresses SNAI1, TCF4, TWIST1, FOXC2, IL1RN, MMP2, SOX10, WNT11, MMP3, PDGFRB, or JAG1. In some embodiments, the cancer a breast cancer (e.g., a triple negative breast cancer) and overexpresses JAG1.

In an aspect, the disclosure provides a method of reducing the expression of PRAME and/or PROS1 in a subject in need thereof, the subject having a high metastatic risk and expressing PRAME and/or PROS1, the method including administering to the subject an effective amount of an agent that reduces the level and/or activity of BRG1 and/or BRM.

In some embodiments, the method further includes surgical intervention, radiation therapy, or chemotherapy to treat the cancer in the subject.

In some embodiments, the administering step is performed after or in conjunction with the surgical intervention, radiation therapy, or chemotherapy.

In some embodiments, the administering step is performed as an adjuvant therapy.

In some embodiments, the agent is administered at a total dose of 2.5-10 mg daily or 2.5-10 mg every other day. In some embodiments, the agent is administered in a total dose of 2.5-7.5 mg daily or 2.5-7.5 mg every other day. In some embodiments, the agent is administered in a total dose of 2.5-5.0 mg daily or 2.5-5.0 mg every other day. In some embodiments, the agent is administered in a total dose of 5.0-7.5 mg daily or 5.0-7.5 mg every other day. In some embodiments, the agent is administered in a total dose of 2.5 mg daily or 2.5 mg every other day. In some embodiments, the agent is administered in a total dose of 5.0 mg daily or 5.0 mg every other day. In some embodiments, the agent is administered in a total dose of 7.5 mg daily or 7.5 mg every other day. In some embodiments, the agent is administered in a total dose of 10 mg daily or 10 mg every other day. In some embodiments, the agent is administered to the subject once daily.

In some embodiments, the agent is administered to the subject once every other day. In some embodiments, the agent is administered orally. In some embodiments, the agent is administered in a unit dosage form selected from a capsule or a tablet.

In some embodiments, if the subject experiences dose limiting toxicity following administration of the agent, the method further includes interrupting administration of the agent.

In some embodiments, the administration of the agent is interrupted for a duration sufficient for the dose limiting toxicity to resolve. In some embodiments, after the dose limiting toxicity is resolved, the method further includes resuming administration of the agent. In some embodiments, the dose limiting toxicity is characterized by keratitis. In some embodiments, the dose limiting toxicity is characterized by Grade 3 keratitis. In some embodiments, the dose limiting toxicity is characterized by hyperbilirubinemia. In some embodiments, the dose limiting toxicity is characterized by Grade 3 hyperbilirubinemia.

In some embodiments, the subject is a human.

In some embodiments, the method decreases metastasis to the liver and brain.

In some embodiments, the method reduces cancer tumor growth in the subject compared to a subject that is not administered the agent.

In some embodiments, the method suppresses metastatic progression of cancer in the subject compared to a subject that is not administered the agent. In some embodiments, the method suppresses metastatic colonization of cancer in the subject compared to a subject that is not administered the agent.

In an aspect, the disclosure provides a method for inhibiting proliferation or growth of cancer stem cells or cancer initiating cells, including contacting the cell with an agent that reduces the level and/or activity of BRG1 and/or BRM in an amount sufficient to inhibit proliferation or growth of the cell.

In some embodiments, the agent is a small molecule inhibitor of BRG1 and/or BRM, e.g., a small molecule inhibitor or a degrader of BRG1 and/or BRM.

In some embodiments, the small molecule BRG1 and/or BRM inhibitor is a compound, or pharmaceutically acceptable salt thereof, having the structure of Formula I:

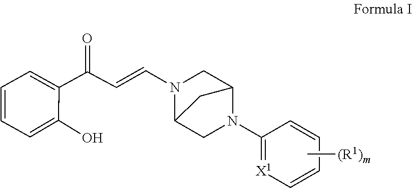

where m is 0, 1, 2, 3, or 4;
X¹ is N or CH; and
each R¹ is, independently, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino.

In some embodiments, the small molecule BRG1 and/or BRM inhibitor is a compound, or pharmaceutically acceptable salt thereof, having the structure of Formula II:

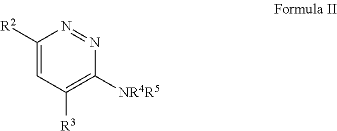

where R² is phenyl that is substituted with hydroxy and that is optionally substituted with one or more groups independently selected from the group consisting of halo, cyano, trifluoromethyl, trifluoromethoxy, C₁₋₃ alkyl, and C₁₋₃ alkoxy;
R³ is selected from the group consisting of —R^{a}, —O—R^{a}, —N(R^{a})2, —S(O)₂R^{a}, and —C(O)—N(R^{a})₂;
each R^{a} is, independently, selected from the group consisting of hydrogen, C₁₋₆ alkyl, C₂₋₆ alkenyl, C₂₋₆ alkynyl, 3-15 membered carbocyclyl, and 3-15 membered heterocyclyl, where each C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{b}, oxo, halo, —NO2, —N(R^{b})₂, —CN, —C(O)—N(R^{b})₂, —S(O)—N(R^{b})₂, —S(O)₂—N(R^{b})₂, —O—R^{b}, —S—R^{b}, —O—C(O)—R^{b}, —C(O)—R^{b}, —C(O)—OR^{b}, —S(O)—R^{b}, S(O)2-R^{b}, —N(R^{b})—C(O)—R^{b}, —N(R^{b})—S(O)—R^{b}, —N(R^{b})—C(O)—N(R^{b})₂, and —N(R^{b})—S(O)₂—R^{b};
each R^{b} is, independently, selected from the group consisting of hydrogen, C₁₋₆ alkyl, C₂₋₆ alkenyl, C₂₋₆ alkynyl, C₁₋₆ alkoxy, 3-15 membered carbocyclyl, and 3-15 membered heterocyclyl, where each C₁₋₆ alkyl, C₂₋₆ alkenyl, C₂₋₆ alkynyl, C₁₋₆ alkoxy, 3-15 membered carbocyclyl, and 3-15 membered heterocyclyl is optionally substituted with one or more groups independently selected from R^{c}; or two R^{b} are taken together with the nitrogen to which they are attached to form a heterocyclyl that is optionally substituted with one or more groups independently selected from the group consisting of oxo, halo and C₁₋₃ alkyl that is optionally substituted with one or more groups independently selected from the group consisting of oxo and halo;
each R^{c} is, independently, selected from the group consisting of oxo, halo, —NO2, —N(R^{d})₂, —CN, —C(O)—N(R^{d})₂, —S(O)—N(R^{d})₂, —S(O)₂—N(R^{d})₂, —S—R^{d}, —O—C(O)—R^{d}, —C(O)—R^{d}, —C(O)—OR d, —S(O)— R^{d}, —S(O)₂—R^{d}, —N(R^{d})—C(O)—R^{d}, —N(R^{d})—S(O)— R^{d}, —N(R^{d})—C(O)—N(R^{d})₂, —N(R^{d})—S(O)₂— R^{d}, C₁₋₆ alkyl, C₂₋₆ alkenyl, C₂₋₆ alkynyl, 3-15 membered carbocyclyl, and 3-15 membered heterocyclyl, where any C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{d}, oxo, halo, —NO₂, —N(R^{d})₂, —CN, —C(O)—N(R^{d})₂, —S(O)—N(R^{d})₂, —S(O)₂—N(R^{d})₂, —OR^{d}, —S—R^{d}, —O—C(O)—R^{d}, —C(O)—R^{d}, —C(O)—R^{d}, —S(O)—R^{d}, —S(O)₂—R^{d}, —N(R^{d})—C(O)—R^{d}, —N(R^{d})—S(O)—R^{d}, —N(R^{d})—C(O)—N(R^{d})₂, and —N(R^{d})—S(O)₂—R^{d};
each R^{d} is, independently, selected from the group consisting of hydrogen, C₁₋₆ alkyl, C₂₋₆ alkenyl, C₂₋₆ alkynyl, carbocyclyl, and carbocyclyl(C₁₋₃ alkyl)-;
R⁴ is H, C₁₋₆ alkyl, or —C(═O)—C₁₋₆ alkyl; and
R⁵ is H or C₁₋₆ alkyl.

Compounds of Formula II may be synthesized by methods known in the art, e.g., those described in U.S. Patent Publication No. 2018/0086720, the synthetic methods of which are incorporated by reference.

In some embodiments, the small molecule BRG1 and/or BRM inhibitor is a compound, or pharmaceutically acceptable salt thereof, having the structure of Formula III:

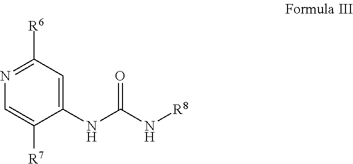

where R⁶ is halo, e.g., fluoro or chloro;
R⁷ is hydrogen, optionally substituted amino, or optionally substituted C₁₋₆ alkyl; and
R⁸ is optionally substituted C₆₋₁₀ aryl or optionally substituted C₂₋₉ heteroaryl.

In some embodiments, the small molecule BRG1 and/or BRM inhibitor is a compound, or pharmaceutically acceptable salt thereof, having the structure of any one of compounds 1-16:

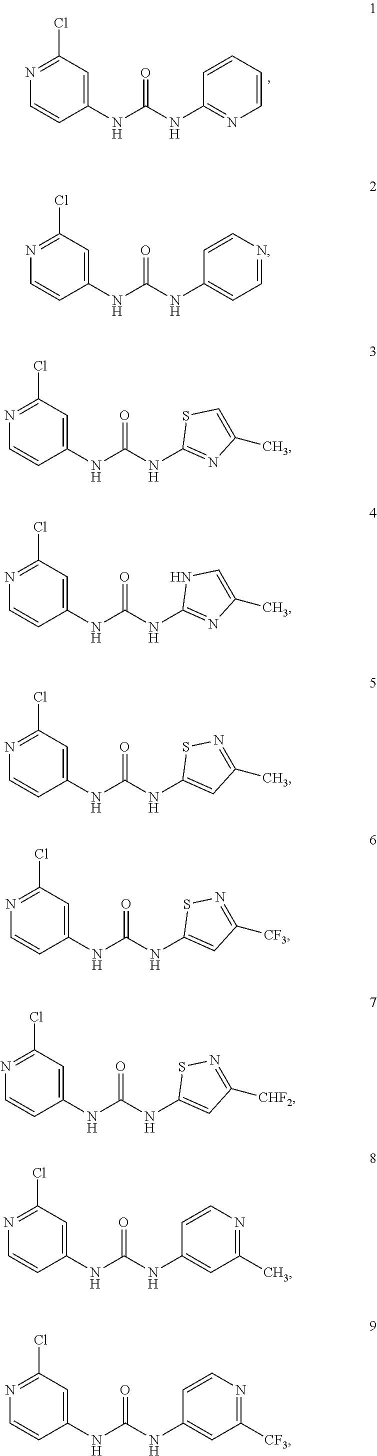

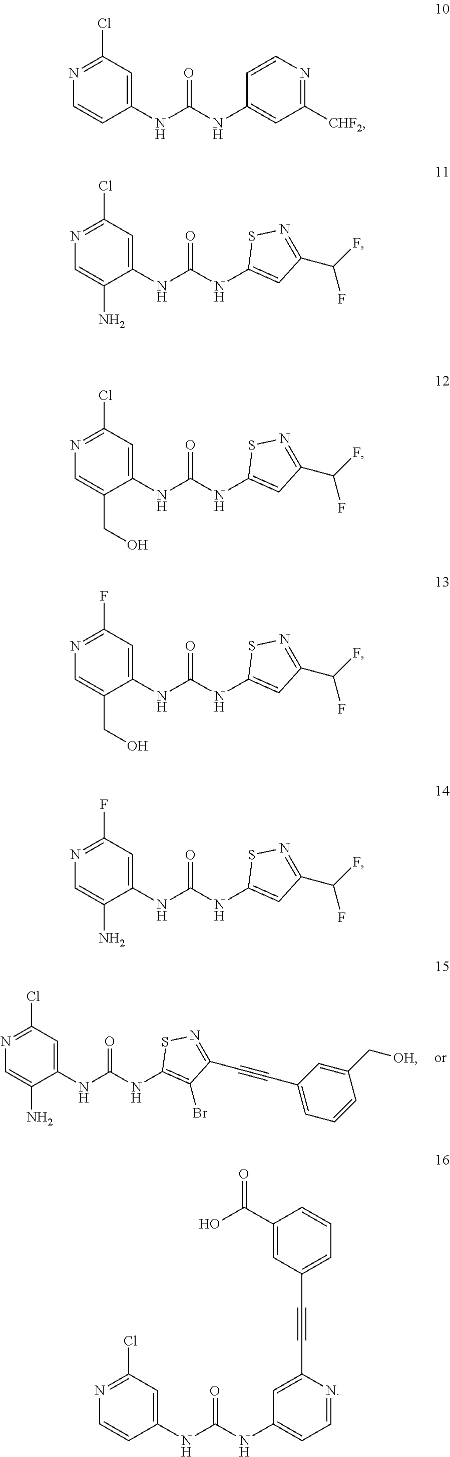

In some embodiments, the small molecule compound, or a pharmaceutically acceptable salt thereof is a degrader. In some embodiments, the degrader has the structure of Formula IV:

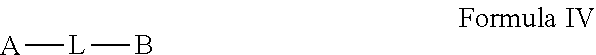

where A is a BRG1 and/or BRM binding moiety; L is a linker; and B is a degradation moiety, or a pharmaceutically acceptable salt thereof. In some embodiments, the degradation moiety is a ubiquitin ligase moiety. In some embodiments, the ubiquitin ligase binding moiety includes Cereblon ligands, IAP (Inhibitors of Apoptosis) ligands, mouse double minute 2 homolog (MDM2), hydrophobic tag, or von Hippel-Lindau ligands, or derivatives or analogs thereof.

In some embodiments, A includes the structure of any one of Formula I-III, or any one of compounds 1-16.

In some embodiments, the hydrophobic tag includes a diphenylmethane, adamantine, or tri-Boc arginine, i.e., the hydrophobic tag includes the structure:

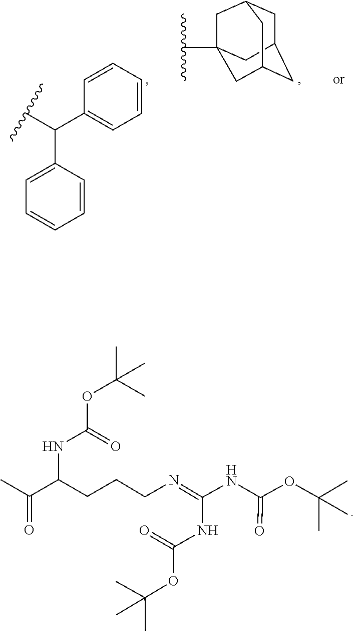 [SMILES: *C(c1ccccc1)c1ccccc1 ; *C12CC3CC(CC(C3)C1)C2 ; CC(=O)C(CCCN=C(NC(=O)OC(C)(C)C)NC(=O)OC(C)(C)C)NC(=O)OC(C)(C)C]

In some embodiments, the ubiquitin ligase binding moiety includes the structure of Formula A:

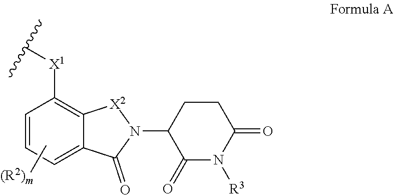

where X¹ is CH₂, O, S, or NR¹, where R¹ is H, optionally substituted C₁-C₆ alkyl, or optionally substituted C₁-C₆ heteroalkyl; X² is C═O, CH₂, or

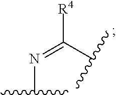

R³ and R⁴ are, independently, H, optionally substituted C₁-C₆ alkyl, or optionally substituted C₁-C₆ heteroalkyl; m is 0, 1, 2, 3, or 4; and each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,
or a pharmaceutically acceptable salt thereof.

In some embodiments, the ubiquitin ligase binding moiety includes the structure:

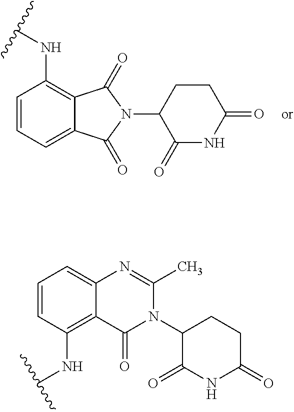 [SMILES: *Nc1cccc2c1C(=O)N(C1CCC(=O)NC1=O)C2=O ; *Nc1cccc2nc(C)n(C3CCC(=O)NC3=O)c(=O)c12]

or is a derivative or an analog thereof, or a pharmaceutically acceptable salt thereof.

In some embodiments, the ubiquitin ligase binding moiety includes the structure of Formula B:

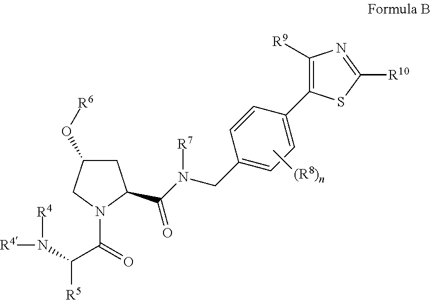

where each R⁴, R^{4′}, and R⁷ is, independently, H, optionally substituted C₁-C₆ alkyl, or optionally substituted C₁-C₆ heteroalkyl; R⁵ is optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R⁶ is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; n is 0, 1, 2, 3, or 4; each R⁸ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and each R⁹ and R¹⁰ is, independently, H, halogen, optionally substituted C₁-C₆ alkyl, or optionally substituted C₆-C₁₀ aryl, where R^{4′} or R⁵ includes a bond to the linker, or a pharmaceutically acceptable salt thereof.

In some embodiments, the ubiquitin ligase binding moiety includes the structure:

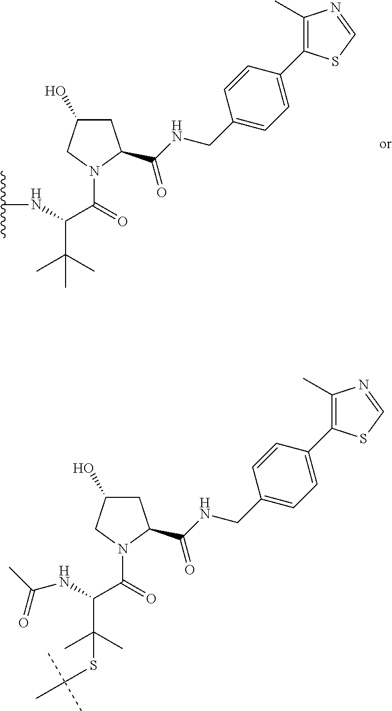

or is a derivative or analog thereof, or a pharmaceutically acceptable salt thereof.

In some embodiments, the ubiquitin ligase binding moiety includes the structure of Formula C:

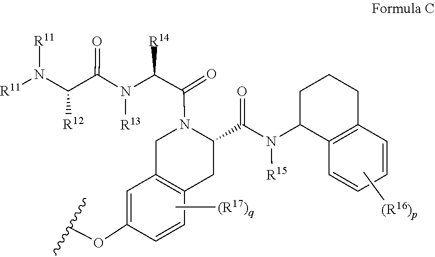

where each R¹¹, R¹³, and R¹⁵ is, independently, H, optionally substituted C₁-C₆ alkyl, or optionally substituted C₁-C₆ heteroalkyl; R¹² is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R¹⁴ is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; p is 0, 1, 2, 3, or 4; each R¹⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; q is 0, 1, 2, 3, or 4; and each R¹⁷ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

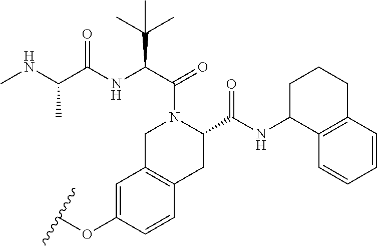 [SMILES: *Oc1ccc2c(c1)CN(C(=O)[C@@H](NC(=O)[C@H](C)NC)C(C)(C)C)[C@H](C(=O)NC1CCCc3ccccc31)C2]

or is a derivative or an analog thereof, or a pharmaceutically acceptable salt thereof.

In some embodiments, the ubiquitin ligase binding moiety includes the structure of Formula D:

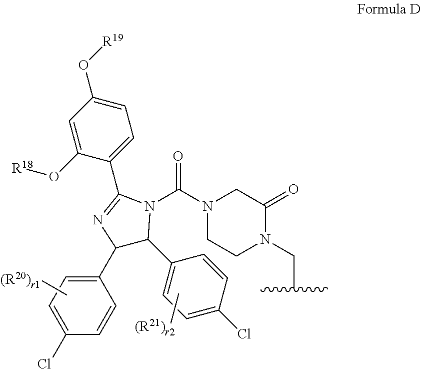

where each R¹⁸ and R¹⁹ is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; r1 is 0, 1, 2, 3, or 4; each R²⁰ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; r2 is 0, 1, 2, 3, or 4; and each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

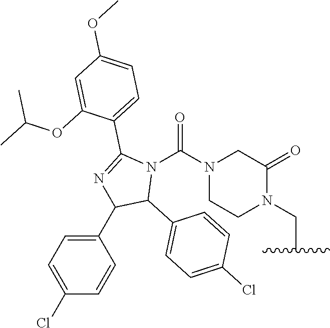 [SMILES: *CN1CCN(C(=O)N2C(c3ccc(OC)cc3OC(C)C)=NC(c3ccc(Cl)cc3)C2c2ccc(Cl)cc2)CC1=O]

or is a derivative or an analog thereof, or a pharmaceutically acceptable salt thereof.

In some embodiments, the linker has the structure of Formula V:

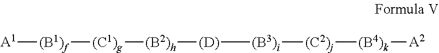

where A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, D is optionally substituted C₂-C₁₀ polyethylene glycol. In some embodiments, C¹ and C² are each, independently, a carbonyl or sulfonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen or optionally substituted C₁₋₄ alkyl. In some embodiments, B¹, B², B³, and B⁴ each, independently, is selected from optionally substituted C₁-C₂ alkyl or optionally substituted C₁-C₃ heteroalkyl. In some embodiments, j is 0. In some embodiments, k is 0. In some embodiments, j and k are each, independently, 0. In some embodiments, f, g, h, and i are each, independently, 1.

In some embodiments, the linker of Formula V has the structure of Formula Va:

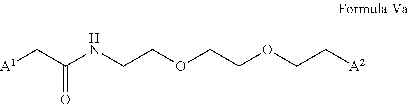

where A¹ is a bond between the linker and A, and A² is a bond between B and the linker.

In some embodiments, D is optionally substituted C₁₋₁₀ alkyl. In some embodiments, C¹ and C² are each, independently, a carbonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}, where R^{N} is hydrogen or optionally substituted C₁₋₄ alkyl. In some embodiments, B¹, B², B³, and B⁴ each, independently, is selected from optionally substituted C₁-C₂ alkyl, O, S, S(O)₂, and NR^{N}, where R^{N} is hydrogen or optionally substituted C₁₋₄ alkyl. In some embodiments, B¹ and B⁴ each, independently, is optionally substituted C₁-C₂ alkyl. In some embodiments, B¹ and B⁴ each, independently, is C₁ alkyl. In some embodiments, B² and B⁴ each, independently, is NR^{N}, where R^{N} is hydrogen or optionally substituted C₁₋₄ alkyl. In some embodiments, B² and B⁴ each, independently, is NH. In some embodiments, f, g, h, l, j, and k are each, independently, 1.

In some embodiments, the linker of Formula V has the structure of Formula Vb:

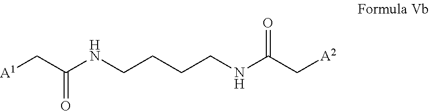

where A¹ is a bond between the linker and A, and A² is a bond between B and the linker.

In some embodiments, the agent that reduces the level and/or activity of BRG1 and/or BRM in a cell is a small molecule compound, an antibody, an enzyme, and/or a polynucleotide.

In some embodiments, the agent that reduces the level and/or activity of BRG1 and/or BRM in a cell is an enzyme, e.g., a clustered regularly interspaced short palindromic repeats (CRISPR)-associated protein such as CRISPR-associated protein 9 (Cas9), CRISPR-associated protein 12a (Cas12a), a zinc finger nuclease (ZFN), a transcription activator-like effector nuclease (TALEN), or a meganuclease.

In some embodiments, the agent that reduces the level and/or activity of BRG1 and/or BRM in a cell is a polynucleotide, e.g., an antisense nucleic acid, a short interfering RNA (siRNA), a short hairpin RNA (shRNA), a microRNA (miRNA), a CRISPR/Cas 9 nucleotide, or a ribozyme.

In some embodiments, the agent inhibits BRG1.

In some embodiments, the agent inhibits epithelial to mesenchymal transition.

In some embodiments, the agent is a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof.

In some embodiments, the compound is:

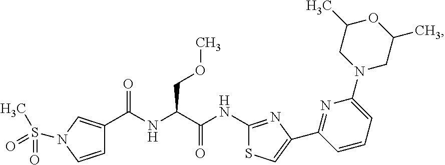 [SMILES: COC[C@H](NC(=O)c1ccn(S(C)(=O)=O)c1)C(=O)Nc1nc(-c2cccc(N3CC(C)OC(C)C3)n2)cs1]

or a pharmaceutically acceptable salt thereof.

In some embodiments, the compound is:

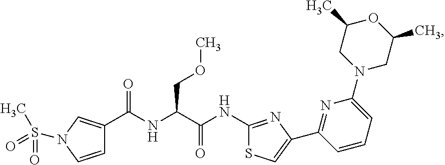 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

or a pharmaceutically acceptable salt thereof.

In some embodiments, the compound is:

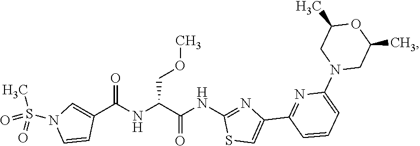 [SMILES: COC[C@@H](NC(=O)c1ccn(S(C)(=O)=O)c1)C(=O)Nc1nc(-c2cccc(N3C[C@@H](C)O[C@@H](C)C3)n2)cs1]

or a pharmaceutically acceptable salt thereof.

### Chemical Terms

For any of the following chemical definitions, a number following an atomic symbol indicates that total number of atoms of that element that are present in a particular chemical moiety. As will be understood, other atoms, such as hydrogen atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, where each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), where each of these recited R^{N1} groups can be optionally substituted; or two R^{N1} combine to form an alkylene or heteroalkylene, and where each R^{N2} is, independently, H, alkyl, or aryl. The amino groups of the compounds described herein can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group.

Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring and containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂—C heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, where no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the disclosure can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the disclosure may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system.

Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HBRM-associated factors complex in a human cell.

As used herein, the term “BAF complex-related disorder” refers to a disorder that is caused or affected by the level of activity of a BAF complex.

As used herein, the term “BRG1” refers to ATP-dependent chromatin remodeler SMARCA4. BRG1 is a component of the BAF complex, a SWI/SNF ATPase chromatin remodeling complex. Human BRG1 is encoded by the SMARCA4 gene on chromosome 19, a nucleic acid sequence of which is set forth in SEQ ID NO: 1 (GenBank Accession No.: NM_001128849.1 (mRNA); www.ncbi.nlm.nih.gov/nuccore/NM_001128849.1 ?report=fasta).

GGCGGGGGAGGCGCCGGGAAGTCGACGGCGCCGGCGGCTCCTGCAGGAGGCCACTGTCTGCAGCTCCCGT
GAAGATGTCCACTCCAGACCCACCCCTGGGCGGAACTCCTCGGCCAGGTCCTTCCCCGGGCCCTGGCCCT
TCCCCTGGAGCCATGCTGGGCCCTAGCCCGGGTCCCTCGCCGGGCTCCGCCCACAGCATGATGGGGCCCA
GCCCAGGGCCGCCCTCAGCAGGACACCCCATCCCCACCCAGGGGCCTGGAGGGTACCCTCAGGACAACAT
GCACCAGATGCACAAGCCCATGGAGTCCATGCATGAGAAGGGCATGTCGGACGACCCGCGCTACAACCAG
ATGAAAGGAATGGGGATGCGGTCAGGGGGCCATGCTGGGATGGGGCCCCCGCCCAGCCCCATGGACCAGC
ACTCCCAAGGTTACCCCTCGCCCCTGGGTGGCTCTGAGCATGCCTCTAGTCCAGTTCCAGCCAGTGGCCC
GTCTTCGGGGCCCCAGATGTCTTCCGGGCCAGGAGGTGCCCCGCTGGATGGTGCTGACCCCCAGGCCTTG
GGGCAGCAGAACCGGGGCCCAACCCCATTTAACCAGAACCAGCTGCACCAGCTCAGAGCTCAGATCATGG
CCTACAAGATGCTGGCCAGGGGGCAGCCCCTCCCCGACCACCTGCAGATGGCGGTGCAGGGCAAGCGGCC
GATGCCCGGGATGCAGCAGCAGATGCCAACGCTACCTCCACCCTCGGTGTCCGCAACAGGACCCGGCCCT
GGCCCTGGCCCTGGCCCCGGCCCGGGTCCCGGCCCGGCACCTCCAAATTACAGCAGGCCTCATGGTATGG
GAGGGCCCAACATGCCTCCCCCAGGACCCTCGGGCGTGCCCCCCGGGATGCCAGGCCAGCCTCCTGGAGG
GCCTCCCAAGCCCTGGCCTGAAGGACCCATGGCGAATGCTGCTGCCCCCACGAGCACCCCTCAGAAGCTG
ATTCCCCCGCAGCCAACGGGCCGCCCTTCCCCCGCGCCCCCTGCCGTCCCACCCGCCGCCTCGCCCGTGA
TGCCACCGCAGACCCAGTCCCCCGGGCAGCCGGCCCAGCCCGCGCCCATGGTGCCACTGCACCAGAAGCA
GAGCCGCATCACCCCCATCCAGAAGCCGCGGGGCCTCGACCCTGTGGAGATCCTGCAGGAGCGCGAGTAC
AGGCTGCAGGCTCGCATCGCACACCGAATTCAGGAACTTGAAAACCTTCCCGGGTCCCTGGCCGGGGATT
TGCGAACCAAAGCGACCATTGAGCTCAAGGCCCTCAGGCTGCTGAACTTCCAGAGGCAGCTGCGCCAGGA
GGTGGTGGTGTGCATGCGGAGGGACACAGCGCTGGAGACAGCCCTCAATGCTAAGGCCTACAAGCGCAGC
AAGCGCCAGTCCCTGCGCGAGGCCCGCATCACTGAGAAGCTGGAGAAGCAGCAGAAGATCGAGCAGGAGC
GCAAGCGCCGGCAGAAGCACCAGGAATACCTCAATAGCATTCTCCAGCATGCCAAGGATTTCAAGGAATA
TCACAGATCCGTCACAGGCAAAATCCAGAAGCTGACCAAGGCAGTGGCCACGTACCATGCCAACACGGAG
CGGGAGCAGAAGAAAGAGAACGAGCGGATCGAGAAGGAGCGCATGCGGAGGCTCATGGCTGAAGATGAGG
AGGGGTACCGCAAGCTCATCGACCAGAAGAAGGACAAGCGCCTGGCCTACCTCTTGCAGCAGACAGACGA
GTACGTGGCTAACCTCACGGAGCTGGTGCGGCAGCACAAGGCTGCCCAGGTCGCCAAGGAGAAAAAGAAG
AAAAAGAAAAAGAAGAAGGCAGAAAATGCAGAAGGACAGACGCCTGCCATTGGGCCGGATGGCGAGCCTC
TGGACGAGACCAGCCAGATGAGCGACCTCCCGGTGAAGGTGATCCACGTGGAGAGTGGGAAGATCCTCAC
AGGCACAGATGCCCCCAAAGCCGGGCAGCTGGAGGCCTGGCTCGAGATGAACCCGGGGTATGAAGTAGCT
CCGAGGTCTGATAGTGAAGAAAGTGGCTCAGAAGAAGAGGAAGAGGAGGAGGAGGAAGAGCAGCCGCAGG
CAGCACAGCCTCCCACCCTGCCCGTGGAGGAGAAGAAGAAGATTCCAGATCCAGACAGCGATGACGTCTC
TGAGGTGGACGCGCGGCACATCATTGAGAATGCCAAGCAAGATGTCGATGATGAATATGGCGTGTCCCAG
GCCCTTGCACGTGGCCTGCAGTCCTACTATGCCGTGGCCCATGCTGTCACTGAGAGAGTGGACAAGCAGT
CAGCGCTTATGGTCAATGGTGTCCTCAAACAGTACCAGATCAAAGGTTTGGAGTGGCTGGTGTCCCTGTA
CAACAACAACCTGAACGGCATCCTGGCCGACGAGATGGGCCTGGGGAAGACCATCCAGACCATCGCGCTC
ATCACGTACCTCATGGAGCACAAACGCATCAATGGGCCCTTCCTCATCATCGTGCCTCTCTCAACGCTGT
CCAACTGGGCGTACGAGTTTGACAAGTGGGCCCCCTCCGTGGTGAAGGTGTCTTACAAGGGATCCCCAGC
AGCAAGACGGGCCTTTGTCCCCCAGCTCCGGAGTGGGAAGTTCAACGTCTTGCTGACGACGTACGAGTAC
ATCATCAAAGACAAGCACATCCTCGCCAAGATCCGTTGGAAGTACATGATTGTGGACGAAGGTCACCGCA
TGAAGAACCACCACTGCAAGCTGACGCAGGTGCTCAACACGCACTATGTGGCACCCCGCCGCCTGCTGCT
GACGGGCACACCGCTGCAGAACAAGCTTCCCGAGCTCTGGGCGCTGCTCAACTTCCTGCTGCCCACCATC
TTCAAGAGCTGCAGCACCTTCGAGCAGTGGTTTAACGCACCCTTTGCCATGACCGGGGAAAAGGTGGACC
TGAATGAGGAGGAAACCATTCTCATCATCCGGCGTCTCCACAAAGTGCTGCGGCCCTTCTTGCTCCGACG
ACTCAAGAAGGAAGTCGAGGCCCAGTTGCCCGAAAAGGTGGAGTACGTCATCAAGTGCGACATGTCTGCG
CTGCAGCGAGTGCTCTACCGCCACATGCAGGCCAAGGGCGTGCTGCTGACTGATGGCTCCGAGAAGGACA
AGAAGGGCAAAGGCGGCACCAAGACCCTGATGAACACCATCATGCAGCTGCGGAAGATCTGCAACCACCC
CTACATGTTCCAGCACATCGAGGAGTCCTTTTCCGAGCACTTGGGGTTCACTGGCGGCATTGTCCAAGGG
CTGGACCTGTACCGAGCCTCGGGTAAATTTGAGCTTCTTGATAGAATTCTTCCCAAACTCCGAGCAACCA
ACCACAAAGTGCTGCTGTTCTGCCAAATGACCTCCCTCATGACCATCATGGAAGATTACTTTGCGTATCG
CGGCTTTAAATACCTCAGGCTTGATGGAACCACGAAGGCGGAGGACCGGGGCATGCTGCTGAAAACCTTC
AACGAGCCCGGCTCTGAGTACTTCATCTTCCTGCTCAGCACCCGGGCTGGGGGGCTCGGCCTGAACCTCC
AGTCGGCAGACACTGTGATCATTTTTGACAGCGACTGGAATCCTCACCAGGACCTGCAAGCGCAGGACCG
AGCCCACCGCATCGGGCAGCAGAACGAGGTGCGTGTGCTCCGCCTCTGCACCGTCAACAGCGTGGAGGAG
AAGATCCTAGCTGCAGCCAAGTACAAGCTCAACGTGGACCAGAAGGTGATCCAGGCCGGCATGTTCGACC
AGAAGTCCTCCAGCCATGAGCGGCGCGCCTTCCTGCAGGCCATCCTGGAGCACGAGGAGCAGGATGAGAG
CAGACACTGCAGCACGGGCAGCGGCAGTGCCAGCTTCGCCCACACTGCCCCTCCGCCAGCGGGCGTCAAC
CCCGACTTGGAGGAGCCACCTCTAAAGGAGGAAGACGAGGTGCCCGACGACGAGACCGTCAACCAGATGA
TCGCCCGGCACGAGGAGGAGTTTGATCTGTTCATGCGCATGGACCTGGACCGCAGGCGCGAGGAGGCCCG
CAACCCCAAGCGGAAGCCGCGCCTCATGGAGGAGGACGAGCTCCCCTCGTGGATCATCAAGGACGACGCG
GAGGTGGAGCGGCTGACCTGTGAGGAGGAGGAGGAGAAGATGTTCGGCCGTGGCTCCCGCCACCGCAAGG
AGGTGGACTACAGCGACTCACTGACGGAGAAGCAGTGGCTCAAGAAAATTACAGGAAAAGATATCCATGA
CACAGCCAGCAGTGTGGCACGTGGGCTACAATTCCAGCGTGGCCTTCAGTTCTGCACACGTGCGTCAAAG
GCCATCGAGGAGGGCACGCTGGAGGAGATCGAAGAGGAGGTCCGGCAGAAGAAATCATCACGGAAGCGCA
AGCGAGACAGCGACGCCGGCTCCTCCACCCCGACCACCAGCACCCGCAGCCGCGACAAGGACGACGAGAG
CAAGAAGCAGAAGAAGCGCGGGCGGCCGCCTGCCGAGAAACTCTCCCCTAACCCACCCAACCTCACCAAG
AAGATGAAGAAGATTGTGGATGCCGTGATCAAGTACAAGGACAGCAGCAGTGGACGTCAGCTCAGCGAGG
TCTTCATCCAGCTGCCCTCGCGAAAGGAGCTGCCCGAGTACTACGAGCTCATCCGCAAGCCCGTGGACTT
CAAGAAGATAAAGGAGCGCATTCGCAACCACAAGTACCGCAGCCTCAACGACCTAGAGAAGGACGTCATG
CTCCTGTGCCAGAACGCACAGACCTTCAACCTGGAGGGCTCCCTGATCTATGAAGACTCCATCGTCTTGC
AGTCGGTCTTCACCAGCGTGCGGCAGAAAATCGAGAAGGAGGATGACAGTGAAGGCGAGGAGAGTGAGGA
GGAGGAAGAGGGCGAGGAGGAAGGCTCCGAATCCGAATCTCGGTCCGTCAAAGTGAAGATCAAGCTTGGC
CGGAAGGAGAAGGCACAGGACCGGCTGAAGGGCGGCCGGCGGCGGCCGAGCCGAGGGTCCCGAGCCAAGC
CGGTCGTGAGTGACGATGACAGTGAGGAGGAACAAGAGGAGGACCGCTCAGGAAGTGGCAGCGAAGAAGA
CTGAGCCCCGACATTCCAGTCTCGACCCCGAGCCCCTCGTTCCAGAGCTGAGATGGCATAGGCCTTAGCA
GTAACGGGTAGCAGCAGATGTAGTTTCAGACTTGGAGTAAAACTGTATAAACAAAAGAATCTTCCATATT
TATACAGCAGAGAAGCTGTAGGACTGTTTGTGACTGGCCCTGTCCTGGCATCAGTAGCATCTGTAACAGC
ATTAACTGTCTTAAAGAGAGAGAGAGAGAATTCCGAATTGGGGAACACACGATACCTGTTTTTCTTTTCC
GTTGCTGGCAGTACTGTTGCGCCGCAGTTTGGAGTCACTGTAGTTAAGTGTGGATGCATGTGCGTCACCG
TCCACTCCTCCTACTGTATTTTATTGGACAGGTCAGACTCGCCGGGGGCCCGGCGAGGGTATGTCAGTGT
CACTGGATGTCAAACAGTAATAAATTAAACCAACAACAAAACGCACAGCCAAAAAAAAA

The term “BRG1” also refers to natural variants of the wild-type human BRG1 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to an amino acid sequence of wild-type BRG1, which is set forth in SEQ ID NO: 2 (UniProt Accession No.: P51532; www.uniprot.org/uniprot/P51532.fasta).

SEQ ID NO: 2
MSTPDPPLGGTPRPGPSPGPGPSPGAMLGPSPGPSPGSAHSMMGPSPGPPSAGHPIPTQG
PGGYPQDNMHQMHKPMESMHEKGMSDDPRYNQMKGMGMRSGGHAGMGPPPSPMDQHSQGY
PSPLGGSEHASSPVPASGPSSGPQMSSGPGGAPLDGADPQALGQQNRGPTPFNQNQLHQL
RAQIMAYKMLARGQPLPDHLQMAVQGKRPMPGMQQQMPTLPPPSVSATGPGPGPGPGPGP
GPGPAPPNYSRPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQSRITPIQKPRG
LDPVEILQEREYRLQARIAHRIQELENLPGSLAGDLRTKATIELKALRLLNFQRQLRQEV
VVCMRRDTALETALNAKAYKRSKRQSLREARITEKLEKQQKIEQERKRRQKHQEYLNSIL
QHAKDFKEYHRSVTGKIQKLTKAVATYHANTEREQKKENERIEKERMRRLMAEDEEGYRK
LIDQKKDKRLAYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYEVAPRSDSEES
GSEEEEEEEEEEQPQAAQPPTLPVEEKKKIPDPDSDDVSEVDARHIIENAKQDVDDEYGV
SQALARGLQSYYAVAHAVTERVDKQSALMVNGVLKQYQIKGLEWLVSLYNNNLNGILADE
MGLGKTIQTIALITYLMEHKRINGPFLIIVPLSTLSNWAYEFDKWAPSVVKVSYKGSPAA
RRAFVPQLRSGKFNVLLTTYEYIIKDKHILAKIRWKYMIVDEGHRMKNHHCKLTQVLNTH
YVAPRRLLLTGTPLQNKLPELWALLNELLPTIFKSCSTFEQWENAPFAMTGEKVDLNEEE
TILIIRRLHKVLRPFLLRRLKKEVEAQLPEKVEYVIKCDMSALQRVLYRHMQAKGVLLTD
GSEKDKKGKGGTKTLMNTIMQLRKICNHPYMFQHIEESFSEHLGFTGGIVQGLDLYRASG
KFELLDRILPKLRATNHKVLLFCQMTSLMTIMEDYFAYRGFKYLRLDGTTKAEDRGMLLK
TFNEPGSEYFIFLLSTRAGGLGLNLQSADTVIIFDSDWNPHQDLQAQDRAHRIGQQNEVR
VLRLCTVNSVEEKILAAAKYKLNVDQKVIQAGMFDQKSSSHERRAFLQAILEHEEQDESR
HCSTGSGSASFAHTAPPPAGVNPDLEEPPLKEEDEVPDDETVNQMIARHEEEFDLEMRMD
LDRRREEARNPKRKPRLMEEDELPSWIIKDDAEVERLTCEEEEEKMFGRGSRHRKEVDYS
DSLTEKQWLKAIEEGTLEEIEEEVRQKKSSRKRKRDSDAGSSTPTTSTRSRDKDDESKKQ
KKRGRPPAEKLSPNPPNLTKKMKKIVDAVIKYKDSSSGRQLSEVFIQLPSRKELPEYYEL
IRKPVDFKKIKERIRNHKYRSLNDLEKDVMLLCQNAQTENLEGSLIYEDSIVLQSVFTSV
RQKIEKEDDSEGEESEEEEEGEEEGSESESRSVKVKIKLGRKEKAQDRLKGGRRRPSRGS
RAKPVVSDDDSEEEQEEDRSGSGSEED.

As used herein, the term “BRG1 activity” refers to the BRG1 enzyme ATPase activity.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

As used herein, the term “BRM” refers to probable global transcription activator SNF2L2. BRM is a component of the BAF complex, a SWI/SNF ATPase chromatin remodeling complex. Human BRM is encoded by the SMARCA2 gene on chromosome 9, a nucleic acid sequence of which is set forth in SEQ 50 ID NO: 3 (GenBank Accession No.: NM_003070.4 www.ncbi.nlm.nih.qov/nuccore/NM 003070.4?report=fasta).

SEQ ID NO: 3
GCGTCTTCCGGCGCCCGCGGAGGAGGCGAGGGTGGGACGCTGGGCGGAGCCCGAGTTTAGGAAGAGGAGG
GGACGGCTGTCATCAATGAAGTCATATTCATAATCTAGTCCTCTCTCCCTCTGTTTCTGTACTCTGGGTG
ACTCAGAGAGGGAAGAGATTCAGCCAGCACACTCCTCGCGAGCAAGCATTACTCTACTGACTGGCAGAGA
CAGGAGAGGTAGATGTCCACGCCCACAGACCCTGGTGCGATGCCCCACCCAGGGCCTTCGCCGGGGCCTG
GGCCTTCCCCTGGGCCAATTCTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGATGGGGTCCACAGACTTCCCA
CAGGAAGGCATGCATCAAATGCATAAGCCCATCGATGGTATACATGACAAGGGGATTGTAGAAGACATCC
ATTGTGGATCCATGAAGGGCACTGGTATGCGACCACCTCACCCAGGCATGGGCCCTCCCCAGAGTCCAAT
GGATCAACACAGCCAAGGTTATATGTCACCACACCCATCTCCATTAGGAGCCCCAGAGCACGTCTCCAGC
CCTATGTCTGGAGGAGGCCCAACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCTGTCCAGCTGCATCAGCTTCG
AGCTCAGATTTTAGCTTATAAAATGCTGGCCCGAGGCCAGCCCCTCCCCGAAACGCTGCAGCTTGCAGTC
CAGGGGAAAAGGACGTTGCCTGGCTTGCAGCAACAACAGCAGCAGCAACAGCAGCAGCAGCAGCAGCAGC
AGCAGCAGCAGCAGCAGCAACAGCAGCCGCAGCAGCAGCCGCCGCAACCACAGACGCAGCAACAACAGCA
GCCGGCCCTTGTTAACTACAACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGCAGCCGCGCAGCCGCCCGCGG
CCGCAGTGCCCGGGCCCTCAGTGCCGCAGCCGGCCCCGGGGCAGCCCTCGCCCGTCCTCCAGCTGCAGCA
GAAGCAGAGCCGCATCAGCCCCATCCAGAAACCGCAAGGCCTGGACCCCGTGGAAATTCTGCAAGAGCGG
GAATACAGACTTCAGGCCCGCATAGCTCATAGGATACAAGAACTGGAAAATCTGCCTGGCTCTTTGCCAC
CAGATTTAAGAACCAAAGCAACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTCTCAACTCCAAAGCATACAAA
CGGAGCAAGCGCCAGACTCTGAGAGAAGCTCGCATGACCGAGAAGCTGGAGAAGCAGCAGAAGATTGAGC
AGGAGAGGAAACGCCGTCAGAAACACCAGGAATACCTGAACAGTATTTTGCAACATGCAAAAGATTTTAA
GGAATATCATCGGTCTGTGGCCGGAAAGATCCAGAAGCTCTCCAAAGCAGTGGCAACTTGGCATGCCAAC
ACTGAAAGAGAGCAGAAGAAGGAGACAGAGCGGATTGAAAAGGAGAGAATGCGGCGACTGATGGCTGAAG
ATGAGGAGGGTTATAGAAAACTGATTGATCAAAAGAAAGACAGGCGTTTAGCTTACCTTTTGCAGCAGAC
CGATGAGTATGTAGCCAATCTGACCAATCTGGTTTGGGAGCACAAGCAAGCCCAGGCAGCCAAAGAGAAG
AAGAAGAGGAGGAGGAGGAAGAAGAAGGCTGAGGAGAATGCAGAGGGTGGGGAGTCTGCCCTGGGACCGG
ATGGAGAGCCCATAGATGAGAGCAGCCAGATGAGTGACCTCCCTGTCAAAGTGACTCACACAGAAACCGG
CAAGGTTCTGTTCGGACCAGAAGCACCCAAAGCAAGTCAGCTGGACGCCTGGCTGGAAATGAATCCTGGT
TATGAAGTTGCCCCTAGATCTGACAGTGAAGAGAGTGATTCTGATTATGAGGAAGAGGATGAGGAAGAAG
AGTCCAGTAGGCAGGAAACCGAAGAGAAAATACTCCTGGATCCAAATAGCGAAGAAGTTTCTGAGAAGGA
TGCTAAGCAGATCATTGAGACAGCTAAGCAAGACGTGGATGATGAATACAGCATGCAGTACAGTGCCAGG
GGCTCCCAGTCCTACTACACCGTGGCTCATGCCATCTCGGAGAGGGTGGAGAAACAGTCTGCCCTCCTAA
TTAATGGGACCCTAAAGCATTACCAGCTCCAGGGCCTGGAATGGATGGTTTCCCTGTATAATAACAACTT
GAACGGAATCTTAGCCGATGAAATGGGGCTTGGAAAGACCATACAGACCATTGCACTCATCACTTATCTG
ATGGAGCACAAAAGACTCAATGGCCCCTATCTCATCATTGTTCCCCTTTCGACTCTATCTAACTGGACAT
ATGAATTTGACAAATGGGCTCCTTCTGTGGTGAAGATTTCTTACAAGGGTACTCCTGCCATGCGTCGCTC
CCTTGTCCCCCAGCTACGGAGTGGCAAATTCAATGTCCTCTTGACTACTTATGAGTATATTATAAAAGAC
AAGCACATTCTTGCAAAGATTCGGTGGAAATACATGATAGTGGACGAAGGCCACCGAATGAAGAATCACC
ACTGCAAGCTGACTCAGGTCTTGAACACTCACTATGTGGCCCCCAGAAGGATCCTCTTGACTGGGACCCC
GCTGCAGAATAAGCTCCCTGAACTCTGGGCCCTCCTCAACTTCCTCCTCCCAACAATTTTTAAGAGCTGC
AGCACATTTGAACAATGGTTCAATGCTCCATTTGCCATGACTGGTGAAAGGGTGGACTTAAATGAAGAAG
AAACTATATTGATCATCAGGCGTCTACATAAGGTGTTAAGACCATTTTTACTAAGGAGACTGAAGAAAGA
AGTTGAATCCCAGCTTCCCGAAAAAGTGGAATATGTGATCAAGTGTGACATGTCAGCTCTGCAGAAGATT
CTGTATCGCCATATGCAAGCCAAGGGGATCCTTCTCACAGATGGTTCTGAGAAAGATAAGAAGGGGAAAG
GAGGTGCTAAGACACTTATGAACACTATTATGCAGTTGAGAAAAATCTGCAACCACCCATATATGTTTCA
GCACATTGAGGAATCCTTTGCTGAACACCTAGGCTATTCAAATGGGGTCATCAATGGGGCTGAACTGTAT
CGGGCCTCAGGGAAGTTTGAGCTGCTTGATCGTATTCTGCCAAAATTGAGAGCGACTAATCACCGAGTGC
TGCTTTTCTGCCAGATGACATCTCTCATGACCATCATGGAGGATTATTTTGCTTTTCGGAACTTCCTTTA
CCTACGCCTTGATGGCACCACCAAGTCTGAAGATCGTGCTGCTTTGCTGAAGAAATTCAATGAACCTGGA
TCCCAGTATTTCATTTTCTTGCTGAGCACAAGAGCTGGTGGCCTGGGCTTAAATCTTCAGGCAGCTGATA
CAGTGGTCATCTTTGACAGCGACTGGAATCCTCATCAGGATCTGCAGGCCCAAGACCGAGCTCACCGCAT
CGGGCAGCAGAACGAGGTCCGGGTACTGAGGCTCTGTACCGTGAACAGCGTGGAGGAAAAGATCCTCGCG
GCCGCAAAATACAAGCTGAACGTGGATCAGAAAGTGATCCAGGCGGGCATGTTTGACCAAAAGTCTTCAA
GCCACGAGCGGAGGGCATTCCTGCAGGCCATCTTGGAGCATGAGGAGGAAAATGAGGAAGAAGATGAAGT
ACCGGACGATGAGACTCTGAACCAAATGATTGCTCGACGAGAAGAAGAATTTGACCTTTTTATGCGGATG
GACATGGACCGGCGGAGGGAAGATGCCCGGAACCCGAAACGGAAGCCCCGTTTAATGGAGGAGGATGAGC
TGCCCTCCTGGATCATTAAGGATGACGCTGAAGTAGAAAGGCTCACCTGTGAAGAAGAGGAGGAGAAAAT
ATTTGGGAGGGGGTCCCGCCAGCGCCGTGACGTGGACTACAGTGACGCCCTCACGGAGAAGCAGTGGCTA
AGGGCCATCGAAGACGGCAATTTGGAGGAAATGGAAGAGGAAGTACGGCTTAAGAAGCGAAAAAGACGAA
GAAATGTGGATAAAGATCCTGCAAAAGAAGATGTGGAAAAAGCTAAGAAGAGAAGAGGCCGCCCTCCCGC
TGAGAAACTGTCACCAAATCCCCCCAAACTGACAAAGCAGATGAACGCTATCATCGATACTGTGATAAAC
TACAAAGATAGGTGTAACGTGGAGAAGGTGCCCAGTAATTCTCAGTTGGAAATAGAAGGAAACAGTTCAG
GGCGACAGCTCAGTGAAGTCTTCATTCAGTTACCTTCAAGGAAAGAATTACCAGAATACTATGAATTAAT
TAGGAAGCCAGTGGATTTCAAAAAAATAAAGGAAAGGATTCGTAATCATAAGTACCGGAGCCTAGGCGAC
CTGGAGAAGGATGTCATGCTTCTCTGTCACAACGCTCAGACGTTCAACCTGGAGGGATCCCAGATCTATG
AAGACTCCATCGTCTTACAGTCAGTGTTTAAGAGTGCCCGGCAGAAAATTGCCAAAGAGGAAGAGAGTGA
GGATGAAAGCAATGAAGAGGAGGAAGAGGAAGATGAAGAAGAGTCAGAGTCCGAGGCAAAATCAGTCAAG
GTGAAAATTAAGCTCAATAAAAAAGATGACAAAGGCCGGGACAAAGGGAAAGGCAAGAAAAGGCCAAATC
GAGGAAAAGCCAAACCTGTAGTGAGCGATTTTGACAGCGATGAGGAGCAGGATGAACGTGAACAGTCAGA
AGGAAGTGGGACGGATGATGAGTGATCAGTATGGACCTTTTTCCTTGGTAGAACTGAATTCCTTCCTCCC
CTGTCTCATTTCTACCCAGTGAGTTCATTTGTCATATAGGCACTGGGTTGTTTCTATATCATCATCGTCT
ATAAACTAGCTTTAGGATAGTGCCAGACAAACATATGATATCATGGTGTAAAAAACACACACATACACAA
ATATTTGTAACATATTGTGACCAAATGGGCCTCAAAGATTCAGATTGAAACAAACAAAAAGCTTTTGATG
GAAAATATGTGGGTGGATAGTATATTTCTATGGGTGGGTCTAATTTGGTAACGGTTTGATTGTGCCTGGT
TTTATCACCTGTTCAGATGAGAAGATTTTTGTCTTTTGTAGCACTGATAACCAGGAGAAGCCATTAAAAG
CCACTGGTTATTTTATTTTTCATCAGGCAATTTTCGAGGTTTTTATTTGTTCGGTATTGTTTTTTTACAC
TGTGGTACATATAAGCAACTTTAATAGGTGATAAATGTACAGTAGTTAGATTTCACCTGCATATACATTT
TTCCATTTTATGCTCTATGATCTGAACAAAAGCTTTTTGAATTGTATAAGATTTATGTCTACTGTAAACA
TTGCTTAATTTTTTTGCTCTTGATTTAAAAAAAAGTTTTGTTGAAAGCGCTATTGAATATTGCAATCTAT
ATAGTGTATTGGATGGCTTCTTTTGTCACCCTGATCTCCTATGTTACCAATGTGTATCGTCTCCTTCTCC
CTAAAGTGTACTTAATCTTTGCTTTCTTTGCACAATGTCTTTGGTTGCAAGTCATAAGCCTGAGGCAAAT
AAAATTCCAGTAATTTCGAAGAATGTGGTGTTGGTGCTTTCCTAATAAAGAAATAATTTAGCTTGACAAA
AAAAAAAAAAAA.

The term “BRM” also refers to natural variants of the wild-type human BRM protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to an amino acid sequence of wild-type BRM, which is set forth in SEQ ID NO: 4 (Uniprot Accession No.: P51531; www.uniprot.org/uniprot/P51531.fasta).

SEQ ID NO: 4
MSTPTDPGAMPHPGPSPGPGPSPGPILGPSPGPGPSPGSVHSMMGPSPGPPSVSHPMPTM
GSTDFPQEGMHQMHKPIDGIHDKGIVEDIHCGSMKGTGMRPPHPGMGPPQSPMDQHSQGY
MSPHPSPLGAPEHVSSPMSGGGPTPPQMPPSQPGALIPGDPQAMSQPNRGPSPFSPVQLH
QLRAQILAYKMLARGQPLPETLQLAVQGKRTLPGLQQQQQQQQQQQQQQQQQQQQQQQPQ
QQPPQPQTQQQQQPALVNYNRPSGPGPELSGPSTPQKLPVPAPGGRPSPAPPAAAQPPAA
AVPGPSVPQPAPGQPSPVLQLQQKQSRISPIQKPQGLDPVEILQEREYRLQARIAHRIQE
LENLPGSLPPDLRTKATVELKALRLLNFQRQLRQEVVACMRRDTTLETALNSKAYKRSKR
QTLREARMTEKLEKQQKIEQERKRRQKHQEYLNSILQHAKDFKEYHRSVAGKIQKLSKAV
ATWHANTEREQKKETERIEKERMRRLMAEDEEGYRKLIDQKKDRRLAYLLQQTDEYVANL
TNLVWEHKQAQAAKEKKKRRRRKKKAEENAEGGESALGPDGEPIDESSQMSDLPVKVTHT
ETGKVLFGPEAPKASQLDAWLEMNPGYEVAPRSDSEESDSDYEEEDEEEESSRQETEEKI
LLDPNSEEVSEKDAKQIIETAKQDVDDEYSMQYSARGSQSYYTVAHAISERVEKQSALLI
NGTLKHYQLQGLEWMVSLYNNNLNGILADEMGLGKTIQTIALITYLMEHKRINGPYLIIV
PLSTLSNWTYEFDKWAPSVVKISYKGTPAMRRSLVPQLRSGKENVLLTTYEYIIKDKHIL
AKIRWKYMIVDEGHRMKNHHCKLTQVLNTHYVAPRRILLTGTPLQNKLPELWALLNFLLP
TIFKSCSTFEQWFNAPFAMTGERVDLNEEETILIIRRLHKVLRPFLLRRLKKEVESQLPE
KVEYVIKCDMSALQKILYRHMQAKGILLTDGSEKDKKGKGGAKTLMNTIMQLRKICNHPY
MFQHIEESFAEHLGYSNGVINGAELYRASGKFELLDRILPKLRATNHRVLLFCQMTSLMT
IMEDYFAFRNFLYLRLDGTTKSEDRAALLKKENEPGSQYFIFLLSTRAGGLGLNLQAADT
VVIFDSDWNPHQDLQAQDRAHRIGQQNEVRVLRLCTVNSVEEKILAAAKYKLNVDQKVIQ
AGMFDQKSSSHERRAFLQAILEHEEENEEEDEVPDDETLNQMIARREEEFDLFMRMDMDR
RREDARNPKRKPRLMEEDELPSWIIKDDAEVERLTCEEEEEKIFGRGSRQRRDVDYSDAL
TEKQWLRAIEDGNLEEMEEEVRLKKRKRRRNVDKDPAKEDVEKAKKRRGRPPAEKLSPNP
PKLTKQMNAIIDTVINYKDRCNVEKVPSNSQLEIEGNSSGRQLSEVFIQLPSRKELPEYY
ELIRKPVDFKKIKERIRNHKYRSLGDLEKDVMLLCHNAQTENLEGSQIYEDSIVLQSVEK
SARQKIAKEEESEDESNEEEEEEDEEESESEAKSVKVKIKLNKKDDKGRDKGKGKKRPNR
GKAKPVVSDFDSDEEQDEREQSEGSGTDDE.

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

The term “CTLA-4 inhibitor,” as used herein, refers to a compound such as an antibody capable of inhibiting the activity of the protein that in humans is encoded by the CTLA4 gene. Known CTLA-4 inhibitors include ipilimumab.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

As used herein, the term “derivative” refers to naturally-occurring, synthetic, and semi-synthetic analogues of a compound, peptide, protein, or other substance described herein. A derivative of a compound, peptide, protein, or other substance described herein may retain or improve upon the biological activity of the original material.

By a “drug resistant” is meant a cancer that does not respond, or exhibits a decreased response to, one or more chemotherapeutic agents (e.g., any agent described herein). A cancer determined to be “resistant” to a drug, as used herein, refers to a cancer that is drug resistant, based on unresponsiveness or decreased responsiveness to a chemotherapeutic agent, or is predicted to be drug resistant based on a prognostic assay (e.g., a gene expression assay).

As used herein, the term “inhibiting BRM” and/or “inhibiting BRG1” refers to blocking or reducing the level or activity of the ATPase catalytic binding domain or the bromodomain of the protein. BRM and/or BRG1 inhibition may be determined using methods known in the art, e.g., a BRM and/or BRG1 ATPase assay, a Nano DSF assay, or a BRM and/or BRG1 Luciferase cell assay.

As used herein, the term “failed to respond to a prior therapy” or “refractory to a prior therapy,” refers to a cancer that progressed despite treatment with the therapy.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

The term “MEK inhibitor,” as used herein, refers to a compound capable of inhibiting the activity of the mitogen-activated protein kinase enzyme MEK1 or MEK2. An MEK inhibitor may be, e.g., selumetinib, binimetinib, ortametinib.

As used herein, “metastatic nodule” refers to an aggregation of tumor cells in the body at a site other than the site of the original tumor.

As used herein, “metastatic cancer” refers to a tumor or cancer in which the cancer cells forming the tumor have a high potential to or have begun to, metastasize, or spread from one location to another location or locations within a subject, via the lymphatic system or via haematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include, but are not limited to, lung cancer (e.g., non-small cell lung cancer), breast cancer, ovarian cancer, colorectal cancer, biliary tract cancer, bladder cancer, brain cancer including glioblastomas and medulloblastomas, cervical cancer, choriocarcinoma, endometrial cancer, esophageal cancer, gastric cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

“Non-metastatic cell migration cancer” as used herein refers to cancers that do not migrate via the lymphatic system or via haematogenous spread.

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., BRG1 and/or BRM). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a BRG1 and/or BRM polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “PD-1 inhibitor,” as used herein, refers to a compound such as an antibody capable of inhibiting the activity of the protein that in humans is encoded by the PDCD1 gene. Known PD-1 inhibitors include nivolumab, pembrolizumab, pidilizumab, BMS 936559, and atezolizumab.

The term “PD-L1 inhibitor,” as used herein, refers to a compound such as an antibody capable of inhibiting the activity of the protein that in humans is encoded by the CD274 gene. Known PD-L1 inhibitors include atezolizumab and durvalumab.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound described herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C.G. Wermuth), Wiley-VCH, 2008. The salts can be prepared in situ during the final isolation and purification of the compounds described herein or separately, e.g., by reacting a free base group with a suitable organic acid.

The compounds of the invention may have ionizable groups so as to be capable of preparation as pharmaceutically acceptable salts. These salts may be, e.g., acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

“Progression-free survival” as used herein, refers to the length of time during and after medication or treatment during which the disease being treated (e.g., cancer) does not get worse.

The term “PKC inhibitor,” as used herein, refers to a compound capable of inhibiting the activity of the protein kinase C. A PKC inhibitor may be, e.g., sotrastaurin or IDE196.

“Proliferation” as used in this application involves reproduction or multiplication of similar forms (cells) due to constituting (cellular) elements.

By “reducing the activity of BRG1 and/or BRM” is meant decreasing the level of an activity related to a BRG1 and/or BRM, or a related downstream effect. A non-limiting example of inhibition of an activity of BRG1 and/or BRM is decreasing the level of a BAF complex (e.g., GBAF) in a cell. The activity level of BRG1 and/or BRM may be measured using any method known in the art. In some embodiments, an agent which reduces the activity of BRG1 and/or BRM is a small molecule BRG1 and/or BRM inhibitor

By “reducing the level of BRG1 and/or BRM” is meant decreasing the level of BRG1 and/or BRM in a cell or subject. The level of BRG1 and/or BRM may be measured using any method known in the art.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the disclosure; a sample from a subject that has been treated by a compound of the disclosure; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the disclosure. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, “slowing the spread of metastasis” refers to reducing or stopping the formation of new loci; or reducing, stopping, or reversing the tumor load.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment. In the context of treating cancer, treatment may include slowing the spread of metastasis and/or extending progression-free survival in a population of treated subjects as compared to a population of untreated subjects. Compounds of the disclosure may also be used to “prophylactically treat” or “prevent” a disorder, for example, in a subject at increased risk of developing the disorder.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a scheme showing classification of uveal melanoma according to its Gene Expression Profile: GEP1 and GEP2. Increase in the expression of PRAME indicates increased metastatic risk. GEP2 uveal melanoma exhibits a higher metastatic risk than GEP1.

FIG. 2 is a bar chart comparing baseline gene expression for GEP2 panel, GEP1 panel, PRAME and BAP1 in three different xenografts, the cell lines being arranged in the order of increasing metastatic risk.

FIG. 3 is a bar chart showing the effect of a 5-day long treatment with Compound A upon MP41 and MP46 xenografts, as measured by the expression level change (Log 2FC) for GEP1 panel, GEP2 panel, and PRAME.

FIG. 4 is a bar chart showing the effect of a 5-day long treatment with Compound A upon MP41 xenograft, as measured by the expression level change (Log 2FC) for BAP1, GDF15, and PROS1.

FIG. 5 is a series of bar charts showing the effect of the treatment with Compound A upon 92.1, MP41, and MP46 xenografts, as measured by the expression level change (Log 2FC) for SOX10, MITF, MLANA, PMEL, TYR, S100A1, and S100B.

FIG. 6 is a bar chart showing expression levels for target engagement markers in 92.1, MP41, and MP46 cell lines upon treatment with a vehicle.

FIG. 7A is a series of images of MP-41 xenograft study, stained for BRG1, BRM, MITF, and SOX10 in tissues from animals treated with a vehicle (left column) and with Compound A (right column).

FIG. 7B is a series of images of MP-41 xenograft study, stained for GP100, MelanA, Tyrosinase, and Cyclin D1 in tissues from animals treated with a vehicle (left column) and with Compound A (right column).

### DETAILED DESCRIPTION

In general, the invention provides methods of managing metastatic cancers, e.g., a method of slowing the spread of a migrating cancer in a subject having a high risk of the spread of the migrating cancer, a method of reducing the rate of tumor seeding of a cancer in a subject having a high risk of tumor seeding of a cancer, a method of reducing or treating metastatic nodule-forming of a cancer in a subject having a high risk of metastatic nodule-forming of a cancer, a method of reducing metastatic risk of a cancer in a subject having a high metastatic risk, a method for treating metastatic cancer in a subject having a high metastatic risk, a method for inhibiting cancer cell invasion or migration in a subject having a high risk of cancer cell invasion or migration, a method of treating GEP2 uveal melanoma in a subject in need thereof, a method of transforming a GEP2 uveal melanoma to a GEP1 uveal melanoma in a subject in need thereof, a method of reducing the expression of PRAME and/or PROS1 in a subject in need thereof, and/or a method for inhibiting proliferation or growth of cancer stem cells or cancer initiating cells. Preferably, the method disclosed herein include the step of administering to the subject an agent that reduces the level and/or activity of BRG1 and/or BRM. The methods disclosed herein may include the step of contacting the cell with an agent that reduces the level and/or activity of BRG1 and/or BRM in an amount sufficient to inhibit proliferation or growth of the cell.

Without wishing to be bound by theory, it is believed that the reduction in the level and/or activity of BRG1 and/or BRM (e.g., using the compound described herein, such as compound A or a pharmaceutically acceptable salt thereof) favorably affects the expression levels of PRAME, PROS1, and/or markers associated with Gene Expression Profile 2 (GEP2), thus indicating a reduction in the metastatic potential of a cancer. A subject may be deemed at high metastatic risk based on the expression of PRAME, PROS1, and/or markers associated with GEP2.

### Method of Treatment

An aspect of the present disclosure relates to methods of treating disorders related to BRG1 loss of function mutations such as cancer (e.g., melanoma, non-small cell lung cancer, small-cell lung cancer, ovarian cancer, breast cancer, esophagogastric cancer, oral cancer, glioblastoma, colon cancer, hematologic cancer, sarcoma, or neuroblastoma) in a subject in need thereof. In some embodiments, the cancer is melanoma. In some embodiments, the melanoma is uveal melanoma, mucosal melanoma, or cutaneous melanoma. In some embodiments, the melanoma is uveal melanoma. In some embodiments, the cancer is a hematologic cancer. In some embodiments, the hematologic cancer is acute myeloid leukemia. In some embodiments, the acute myeloid leukemia is refractory acute myeloid leukemia. In some embodiments, the acute myeloid leukemia is relapsed acute myeloid leukemia. In some embodiments, the cancer is breast cancer. In some embodiments, the breast cancer is an ER positive breast cancer, an ER negative breast cancer, triple positive breast cancer, or triple negative breast cancer. In some embodiments, the breast cancer is a triple negative breast cancer. In some embodiments, the cancer is an oral cancer that is an oral squamous cancer.

In some embodiments, the methods of the present disclosure result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, esophageal cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, hematologic cancer, and penile cancer.

In some embodiments, the cancer is melanoma, non-small cell lung cancer, small-cell lung cancer, ovarian cancer, breast cancer, esophagogastric cancer, oral cancer, glioblastoma, colon cancer, hematologic cancer, sarcoma, or neuroblastoma. In some embodiments, the cancer is melanoma (e.g., uveal melanoma, mucosal melanoma, or cutaneous melanoma). In some embodiments, the cancer is a hematologic cancer (e.g., acute myeloid leukemia (e.g., refractory acute myeloid leukemia or relapsed acute myeloid leukemia.

In some embodiments, the cancer is breast cancer.

In some embodiments, the breast cancer is an ER positive breast cancer, an ER negative breast cancer, triple positive breast cancer, or triple negative breast cancer.

In some embodiments, the breast cancer is a triple negative breast cancer.

In some embodiments, the cancer is an oral cancer that is an oral squamous cancer.

In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the subject or cancer has and/or has been identified as having a BRG1 loss of function mutation. In some embodiments, the subject or cancer has and/or has been identified as having a BRM loss of function mutation.

In some embodiments, the cancer has or has been determined to have one or more BRG1 mutations (e.g., homozygous mutations). In some embodiments, the one or more BRG1 mutations includes a mutation in the ATPase catalytic domain of the protein. In some embodiments, the one or more BRG1 mutations include a deletion at the C-terminus of BRG1.

In some embodiments, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation.

In some embodiments, the cancer has, or has been determined to have, a mutation in GNAQ. In some embodiments, the cancer has, or has been determined to have, a mutation in GNA11. In some embodiments, the cancer has, or has been determined to have, a mutation in PLCB4. In some embodiments, the cancer has, or has been determined to have, a mutation in CYSLTR2. In some embodiments, the cancer has, or has been determined to have, a mutation in BAP1. In some embodiments, the cancer has, or has been determined to have, a mutation in SF3B1. In some embodiments, the cancer has, or has been determined to have, a mutation in EIF1AX. In some embodiments, the cancer has, or has been determined to have, a TFE3 translocation. In some embodiments, the cancer has, or has been determined to have, a TFEB translocation. In some embodiments, the cancer has, or has been determined to have, a MITF translocation. In some embodiments, the cancer has, or has been determined to have, an EZH2 mutation. In some embodiments, the cancer has, or has been determined to have, a SUZ12 mutation. In some embodiments, the cancer has, or has been determined to have, an EED mutation.

In some embodiments, the subject underexpresses (e.g., a sample from the subject is determined to have the following as underexpressed) BAP1, MGP, CXCL14, CLCA2, S100A8, BTG1, SAP130, ARG1, KRT6B, GJA, ID2, EIF1B, S100A9, CRABP2, KRT14, ROBO1, RBM23, TACSTD2, DSC1, SPRR1B, TRIM29, AQP3, TYRP1, PPL, LTA4H, or CST6. In some embodiments, the subject overexpresses (e.g., a sample from the subject is determined to have the following as overexpessed) SPP1. In some embodiments, the cancer underexpresses (e.g., a cancer tissue sample from the subject is determined to have the following as underexpressed) BAP1, MGP, CXCL14, CLCA2, S100A8, BTG1, SAP130, ARG1, KRT6B, GJA, ID2, EIF1B, S100A9, CRABP2, KRT14, ROBO1, RBM23, TACSTD2, DSC1, SPRR1B, TRIM29, AQP3, TYRP1, PPL, LTA4H, or CST6. In some embodiments, the cancer overexpresses (e.g., a cancer tissue sample from the subject is determined to have the following as overexpessed) SPP1.

The dosage of the compounds of the disclosure, and/or compositions comprising a compound of the disclosure, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the disclosure may be administered initially in a suitable dosage that may be adjusted as required, depending on the clinical response.

In some embodiments, an agent of the disclosure is administered at a total dose of 2.5-10 mg daily or 2.5-10 mg every other day. In some embodiments, an agent of the disclosure is administered at a total dose of 5.0-10 mg daily or 5.0-10 mg every other day. In some embodiments, an agent of the disclosure is administered at a total dose of 2.5-5.0 mg daily or 2.5-5.0 mg every other day. In some embodiments, an agent of the disclosure is administered at a total dose of 5.0-7.5 mg daily or 5.0-7.5 mg every other day. In some embodiments, an agent of the disclosure is administered at a total dose of 7.5-10 mg daily or 7.5-10 mg every other day. In some embodiments, an agent of the disclosure is administered at a total dose of 2.5-7.5 mg daily or 2.5-7.5 mg every other day. In some embodiments, an agent of the disclosure is administered at total dose of 2.5 mg daily or 2.5 mg every other day. In some embodiments, an agent of the disclosure is administered at total dose of 5.0 mg daily or 5.0 mg every other day. In some embodiments, an agent of the disclosure is administered at total dose of 7.5 mg daily or 7.5 mg every other day. In some embodiments, an agent of the disclosure is administered at total dose of 10 mg daily or 10 mg every other day.

In some embodiments, an agent of the disclosure is administered once daily. In some embodiments, an agent of the disclosure is administered once every other day.

Administration of an agent of the disclosure may be interrupted (i.e., a dose hold) if the subject exhibits dose limiting toxicity. Dose limiting toxicity is characterized by conditions including, but are not limited to, hyperbilirubinemia (e.g., Grade 3, Grade 4, or Grade 5 hyperbilirubinemia) and keratitis (e.g., Grade 1, Grade 2, Grade 3, Grade 4, or Grade 5 keratitis). If administration of the compound or pharmaceutically acceptable salt of the disclosure is interrupted, its administration may be resumed if, e.g., the one or more signs of dose limiting toxicity is resolved or improved, e.g., Grade 3 hyperbilirubinemia to Grade 1 or Grade 2 hyperbilirubinemia, or Grade 3 keratitis to Grade 1 or Grade 2 keratitis.

In some embodiments of any of methods disclosed herein, the cancer is drug resistant (e.g., the cancer has been determined to be resistant, or likely to be resistant, to chemotherapeutic or cytotoxic agents such as by genetic markers, or is likely to be resistant, to chemotherapeutic or cytotoxic agents such as a cancer that has failed to respond to a chemotherapeutic or cytotoxic agent) and/or has failed to respond to a prior therapy (e.g., a chemotherapeutic or cytotoxic agent, immunotherapy, surgery, radiotherapy, thermotherapy, or photocoagulation, or a combination thereof).

In some embodiments, the cancer is resistant to and/or has failed to respond to vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin® (trastuzumab), Perjeta® (pertuzumab), tamoxifen, Xeloda® (capecitabine), platinum agents such as carboplatin, taxanes such as paclitaxel and docetaxel, ALK inhibitors, MET inhibitors, Alimta (pemetrexed), Abraxane, doxorubicin, gemcitabine, Avastin®, Halaven®, neratinib, a PARP inhibitor, brilanestrant, an mTOR inhibitor, topotecan, Gemzar (gemcitabine HCl), a VEGFR2 inhibitor, a folate receptor antagonist, demcizumab, fosbretabulin, or a PDL1 inhibitor, or combinations thereof.

In some embodiments of any of the foregoing methods, the cancer is resistant to and/or has failed to respond to dacarbazine, temozolomide, cisplatin, treosulfan, fotemustine, IMCgp100, a CTLA-4 inhibitor (e.g., ipilimumab), a PD-1 inhibitor (e.g., nivolumab or pembrolizumab), a PD-L1 inhibitor (e.g., atezolizumab, avelumab, or durvalumab), a rnitogen-activated protein kinase (MEK) inhibitor (e.g., selumetinib, binimetinib, or tametinib), and/or a protein kinase C (PKC) inhibitor (e.g., sotrastaurin or IDE196).

In some embodiments of any of the foregoing methods, the cancer is resistant to and/or failed to respond to a previously administered therapeutic used for the treatment of uveal melanoma, e.g., a MEK inhibitor or PKC inhibitor. For example, in some embodiments, the cancer is resistant to and/or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments of any of the foregoing methods, the method or effective amount reduces the level and/or activity of BRG1 by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference.

In some embodiments of any of the foregoing methods, the method or effective amount reduces the level and/or activity of BRG1 by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference for at least 12 hours (e.g., at least 14 hours, at least 16 hours, at least 18 hours, at least 20 hours, at least 22 hours, at least 24 hours, at least 30 hours, at least 36 hours, at least 48 hours, at least 72 hours, at least 4 days, at least 5 days, at least 6 days, at least 7 days, at least 14 days, at least 21 days, at least 28 days, or more).

In some embodiments of any of the foregoing methods, the method or effective amount reduces the level and/or activity of BRM by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference.

In some embodiments of any of the foregoing methods, the method or effective amount reduces the level and/or activity of BRM by at least 5% (e.g., at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) as compared to a reference for at least 12 hours (e.g., at least 14 hours, at least 16 hours, at least 18 hours, at least 20 hours, at least 22 hours, at least 24 hours, at least 30 hours, at least 36 hours, at least 48 hours, at least 72 hours, at least 4 days, at least 5 days, at least 6 days, at least 7 days, at least 14 days, at least 21 days, at least 28 days, or more).

BRG1 and/or BRM-Reducing Agents

Agents described herein that reduce the level and/or activity of BRG1 and/or BRM in a cell may be an antibody, a protein (such as an enzyme), a polynucleotide, or a small molecule compound. The agents reduce the level of an activity related to BRG1 and/or BRM, or a related downstream effect, or reduce the level of BRG1 and/or BRM in a cell or subject.

In some embodiments, the agent that reduces the level and/or activity of BRG1 and/or BRM in a cell is an enzyme, a polynucleotide, or a small molecule compound such as a small molecule BRG1 and/or BRM inhibitor.

### Antibodies

The agent that reduces the level and/or activity of BRG1 and/or BRM can be an antibody or antigen binding fragment thereof. For example, an agent that reduces the level and/or activity of BRG1 and/or BRM described herein is an antibody that reduces or blocks the activity and/or function of BRG1 and/or BRM through binding to BRG1 and/or BRM.

The making and use of therapeutic antibodies against a target antigen (e.g., BRG1 and/or BRM) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### Polynucleotides

In some embodiments, the agent that reduces the level and/or activity of BRG1 and/or BRM is a polynucleotide. In some embodiments, the polynucleotide is an inhibitory RNA molecule, e.g., that acts by way of the RNA interference (RNAi) pathway. An inhibitory RNA molecule can decrease the expression level (e.g., protein level or mRNA level) of BRG1 and/or BRM. For example, an inhibitory RNA molecule includes a short interfering RNA (siRNA), short hairpin RNA (shRNA), and/or a microRNA (miRNA) that targets full-length BRG1 and/or BRM. A siRNA is a double-stranded RNA molecule that typically has a length of about 19-25 base pairs. A shRNA is a RNA molecule including a hairpin turn that decreases expression of target genes via RNAi. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. Degradation is caused by an enzymatic, RNA-induced silencing complex (RISC).

In some embodiments, the agent that reduces the level and/or activity of BRG1 and/or BRM is an antisense nucleic acid. Antisense nucleic acids include antisense RNA (asRNA) and antisense DNA (asDNA) molecules, typically about 10 to 30 nucleotides in length, which recognize polynucleotide target sequences or sequence portions through hydrogen bonding interactions with the nucleotide bases of the target sequence (e.g., BRG1 and/or BRM). The target sequences may be single- or double-stranded RNA, or single- or double-stranded DNA.

In some embodiments, the polynucleotide decreases the level and/or activity of a negative regulator of function or a positive regulator of function. In other embodiments, the polynucleotide decreases the level and/or activity of an inhibitor of a positive regulator of function.

A polynucleotide of the invention can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity. The polynucleotides mentioned above, may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. These moieties may be attached to the nucleic acid at the 3′ or 5′ ends and may also be attached through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include hydroxyl protecting groups known in the art, including glycols such as polyethylene glycol and tetraethylene glycol. The inhibitory action of the polynucleotide can be examined using a cell-line or animal based gene expression system of the present invention in vivo and in vitro.In some embodiments, the polynucleotide decreases the level and/or activity or function of BRG1 and/or BRM. In embodiments, the polynucleotide inhibits expression of BRG1 and/or BRM. In other embodiments, the polynucleotide increases degradation of BRG1 and/or BRM and/or decreases the stability (i.e., half-life) of BRG1 and/or BRM. The polynucleotide can be chemically synthesized or transcribed in vitro.

Inhibitory polynucleotides can be designed by methods well known in the art. siRNA, miRNA, shRNA, and asRNA molecules with homology sufficient to provide sequence specificity required to uniquely degrade any RNA can be designed using programs known in the art, including, but not limited to, those maintained on websites for Thermo Fisher Scientific, the German Cancer Research Center, and The Ohio State University Wexner Medical Center. Systematic testing of several designed species for optimization of the inhibitory polynucleotide sequence can be routinely performed by those skilled in the art. Considerations when designing interfering polynucleotides include, but are not limited to, biophysical, thermodynamic, and structural considerations, base preferences at specific positions in the sense strand, and homology. The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010. Exemplary inhibitory polynucleotides, for use in the methods of the invention, are provided in Table 1, below. In some embodiments, the inhibitory polynucleotides have a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleic acid sequence of an inhibitory polynucleotide in Table 1. In some embodiments, the inhibitory polynucleotides have a nucleic acid sequence with at least 70% sequence identity (e.g., 70%, 75%, 80%, 85%, 86%, 87%, 88%, 89%, 90%, 91%, 92%, 93%, 94%, 95%, 96%, 97%, 98%, 99%, 99.9% identity, or more) to the nucleic acid sequence of an inhibitory polynucleotide in Table 1.

Construction of vectors for expression of polynucleotides for use in the invention may be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

### Gene Editing

In some embodiments, the agent that reduces the level and/or activity of BRG1 and/or BRM is a component of a gene editing system. For example, the agent that reduces the level and/or activity of BRG1 and/or BRM introduces an alteration (e.g., insertion, deletion (e.g., knockout), translocation, inversion, single point mutation, or other mutation) in BRG1 and/or BRM. In some embodiments, the agent that reduces the level and/or activity of BRG1 and/or BRM is a nuclease. Exemplary gene editing systems include the zinc finger nucleases (ZFNs), Transcription Activator-Like Effector-based Nucleases (TALENs), and the clustered regulatory interspaced short palindromic repeat (CRISPR) system. ZFNs, TALENs, and CRISPR-based methods are described, e.g., in Gaj et al., Trends Biotechnol. 31(7):397-405 (2013).

CRISPR refers to a set of (or system including a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482(7385):331-338 (2012). For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that include a repeat sequence flanked by a spacer. The RNAs serve as guides to direct Cas proteins to silence specific DNA/RNA sequences, depending on the spacer sequence. See, e.g., Horvath et al., Science 327(5962):167-170 (2010); Makarova et al., Biology Direct 1:7 (2006); Pennisi, Science 341(6148):833-836 (2013). In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a BRG1 and/or BRM sequence. In some embodiments, in a CRISPR system for use described herein, e.g., in accordance with one or more methods described herein, the spacers of the CRISPR are derived from a target gene sequence, e.g., from a BRG1 sequence. In some embodiments, in a CRISPR system for use described herein, e.g., in accordance with one or more methods described herein, the spacers of the CRISPR are derived from a target gene sequence, e.g., from a BRM sequence.

In some embodiments, the agent that reduces the level and/or activity of BRG1 and/or BRM includes a guide RNA (gRNA) for use in a CRISPR system for gene editing. Exemplary gRNAs, for use in the methods of the invention, are provided in Table 1, below. In embodiments, the agent that reduces the level and/or activity of BRG1 and/or BRM includes a ZFN, or an mRNA encoding a ZFN, that targets (e.g., cleaves) a nucleic acid sequence (e.g., DNA sequence) of BRG1 and/or BRM. In embodiments, the agent that reduces the level and/or activity of BRG1 and/or BRM includes a TALEN, or an mRNA encoding a TALEN, that targets (e.g., cleaves) a nucleic acid sequence (e.g., DNA sequence) of BRG1 and/or BRM. In embodiments, the agent that reduces the level and/or activity of BRG1 and/or BRM includes a TALEN, or an mRNA encoding a TALEN, that targets (e.g., cleaves) a nucleic acid sequence (e.g., DNA sequence) of BRG1. In embodiments, the agent that reduces the level and/or activity of BRG1 and/or BRM includes a TALEN, or an mRNA encoding a TALEN, that targets (e.g., cleaves) a nucleic acid sequence (e.g., DNA sequence) of BRM.

For example, the gRNA can be used in a CRISPR system to engineer an alteration in a gene (e.g., BRG1 and/or BRM). In other examples, the ZFN and/or TALEN can be used to engineer an alteration in a gene (e.g., BRG1 and/or BRM). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) BRG1 and/or BRM, e.g., the alteration is a negative regulator of function. In yet another example, the alteration corrects a defect (e.g., a mutation causing a defect), in BRG1 and/or BRM. In yet another example, the alteration corrects a defect (e.g., a mutation causing a defect), in BRG1. In yet another example, the alteration corrects a defect (e.g., a mutation causing a defect), in BRM.

In certain embodiments, the CRISPR system is used to edit (e.g., to add or delete a base pair) a target gene, e.g., BRG1 and/or BRM. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a target gene, e.g., BRG1 and/or BRM, thereby blocking an RNA polymerase sterically. In embodiments, the CRISPR system is used to direct Cas to a promoter of a target gene, e.g., BRG1, thereby blocking an RNA polymerase sterically. In embodiments, the CRISPR system is used to direct Cas to a promoter of a target gene, e.g., BRM, thereby blocking an RNA polymerase sterically.

In some embodiments, a CRISPR system can be generated to edit BRG1 and/or BRM using technology described in, e.g., U.S. Publication No. 20140068797; Cong et al., Science 339(6121):819-823 (2013); Tsai, Nature Biotechnol., 32(6):569-576 (2014); and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding BRG1 and/or BRM. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression. In some embodiments, CRISPR-mediated gene activation (CRISPRa) can be used for transcriptional activation, e.g., of one or more genes described herein, e.g., a gene that inhibits BRG1 and/or BRM. In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Biol. 17(1):5-15 (2016), incorporated herein by reference.

### Small Molecule Compounds

In some embodiments of the invention, the agent that reduces the level and/or activity of BRG1 and/or BRM in a cell is a small molecule compound. In some embodiments, the small molecule compound is a structure of Formula I-III.

In some embodiments, the small molecule BRG1 and/or BRM inhibitor is a compound, or pharmaceutically acceptable salt thereof, having the structure of Formula I:

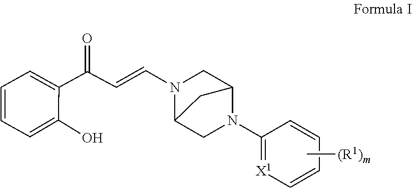

wherein m is 0, 1, 2, 3, or 4;
X¹ is N or CH; and
each R¹ is, independently, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino.

In some embodiments, the small molecule BRG1 and/or BRM inhibitor is a compound, or pharmaceutically acceptable salt thereof, having the structure of Formula II:

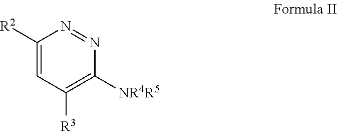

wherein R² is phenyl that is substituted with hydroxy and that is optionally substituted with one or more groups independently selected from the group consisting of halo, cyano, trifluoromethyl, trifluoromethoxy, C₁₋₃ alkyl, and C₁₋₃ alkoxy;
R³ is selected from the group consisting of —R^{a}, —O—R^{a}, —N(R^{a})2, —S(O)₂R^{a}, and —C(O)—N(R^{a})₂;
each R^{a} is, independently, selected from the group consisting of hydrogen, C₁₋₆ alkyl, C₂₋₆ alkenyl, C₂₋₆ alkynyl, 3-15 membered carbocyclyl, and 3-15 membered heterocyclyl, wherein each C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{b}, oxo, halo, —NO2, —N(R^{b})₂, —CN, —C(O)—N(R^{b})₂, —S(O)—N(R^{b})₂, —S(O)₂—N(R^{b})₂, —O—R^{b}, —S—R^{b}, —O—C(O)—R^{b}, —C(O) R^{b}, —C(O)—OR^{b}, —S(O)—R^{b}, —S(O)2-R^{b}, —N(R^{b})—C(O)—R^{b}, —N(R^{b})—S(O)—R^{b}, —N(R^{b})—C(O)—N(R^{b})₂, and —N(R^{b})—S(O)₂—R^{b};
each R^{b} is independently selected from the group consisting of hydrogen, C₁₋₆ alkyl, C₂₋₆ alkenyl, C₂₋₆ alkynyl, C₁₋₆ alkoxy, 3-15 membered carbocyclyl, and 3-15 membered heterocyclyl, wherein each C₁₋₆ alkyl, C₂₋₆ alkenyl, C₂₋₆ alkynyl, C₁₋₆ alkoxy, 3-15 membered carbocyclyl, and 3-15 membered heterocyclyl is optionally substituted with one or more groups independently selected from R^{c}; or two R^{b} are taken together with the nitrogen to which they are attached to form a heterocyclyl that is optionally substituted with one or more groups independently selected from the group consisting of oxo, halo and C₁₋₃ alkyl that is optionally substituted with one or more groups independently selected from the group consisting of oxo and halo;
each R^{c} is independently selected from the group consisting of oxo, halo, —NO2, —N(R^{d})₂, —CN, —C(O)—N(R^{d})₂, —S(O)—N(R^{d})₂, —S(O)₂—N(R^{d})₂, —S—R^{d}, —O—C(O)—R^{d}, —C(O)—R^{d}, —C(O)—OR^{d}, —S(O)— R^{d}, —S(O)₂—R^{d}, —N(R^{d})—C(O)—R^{d}, —N(R^{d})—S(O)— R^{d}, —N(R^{d})—C(O)—N(R^{d})2, —N(R^{d})—S(O)₂— R^{d}, C₁₋₆ alkyl, C₂₋₆ alkenyl, C₂₋₆ alkynyl, 3-15 membered carbocyclyl, and 3-15 membered heterocyclyl, wherein any C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{d}, oxo, halo, —NO₂, —N(R^{d})₂, —CN, —C(O)—N(R^{d})₂, —S(O)—N(R^{d})₂, —S(O)₂—N(R^{d})₂, —OR^{d}, —SR^{d}, —O—C(O)—R^{d}, —C(O)— R^{d}, —C(O)—R^{d}, —S(O)—R^{d}, —S(O)₂—R^{d}, —N(R^{d})—C(O)—R^{d}, —N(R^{d})—S(O)—R^{d}, —N(R^{d})—C(O)—N(R^{d})₂, and —N(R^{d})—S(O)₂—R^{d};
each R^{d} is independently selected from the group consisting of hydrogen, C₁₋₆ alkyl, C₂₋₆ alkenyl, C₂₋₆ alkynyl, carbocyclyl, and carbocyclyl(C₁₋₃ alkyl)-;
R⁴ is H, C₁₋₆ alkyl, or —C(═O)—C₁₋₆ alkyl; and
R⁵ is H or C₁₋₆ alkyl.

Compounds of Formula II may be synthesized by methods known in the art, e.g., those described in U.S. Patent Publication No. 2018/0086720, the synthetic methods of which are incorporated by reference.

In some embodiments, the small molecule BRG1 and/or BRM inhibitor is a compound, or pharmaceutically acceptable salt thereof, having the structure of Formula III:

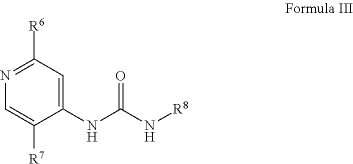

wherein R⁶ is halo, e.g., fluoro or chloro;
R⁷ is hydrogen, optionally substituted amino, or optionally substituted C₁₋₆ alkyl; and
R⁸ is optionally substituted C₆₋₁₀ aryl or optionally substituted C₂₋₉ heteroaryl.

In some embodiments, the small molecule BRG1 and/or BRM inhibitor is a compound, or pharmaceutically acceptable salt thereof, having the structure of any one of compounds 1-16:

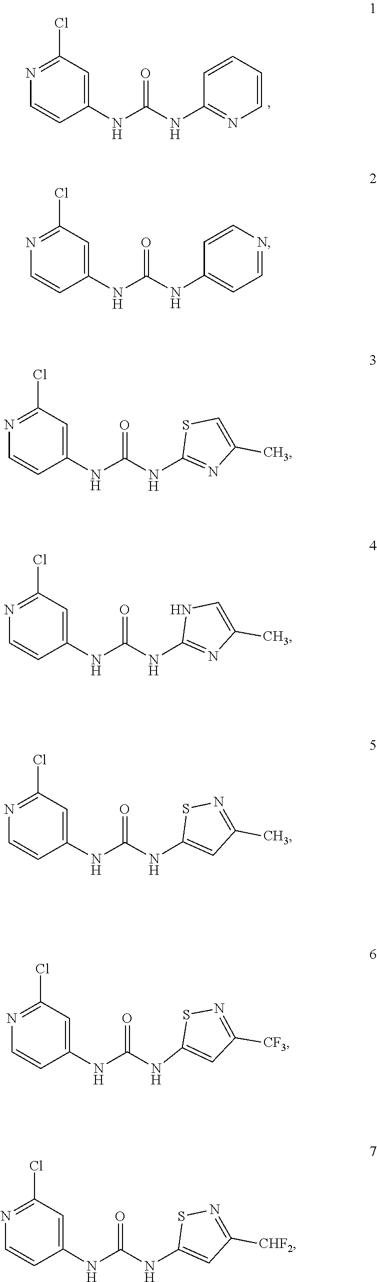

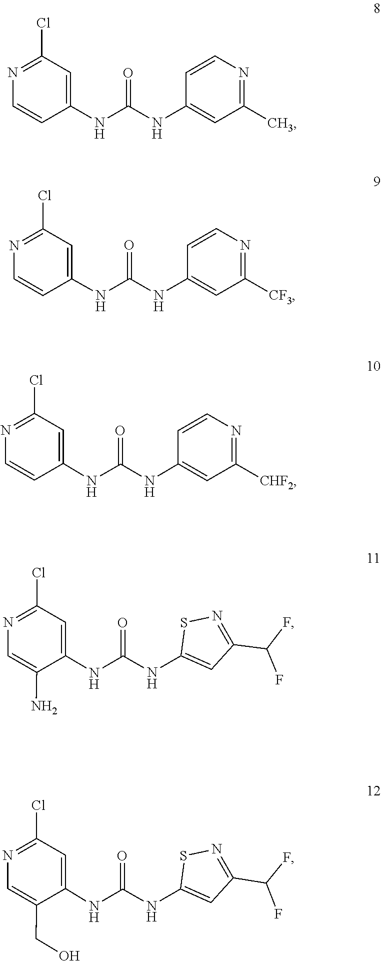 [SMILES: Cc1cc(NC(=O)Nc2ccnc(Cl)c2)ccn1 ; O=C(Nc1ccnc(Cl)c1)Nc1ccnc(C(F)(F)F)c1 ; O=C(Nc1ccnc(Cl)c1)Nc1ccnc(C(F)F)c1 ; Nc1cnc(Cl)cc1NC(=O)Nc1cc(C(F)F)ns1 ; O=C(Nc1cc(C(F)F)ns1)Nc1cc(Cl)ncc1CO]

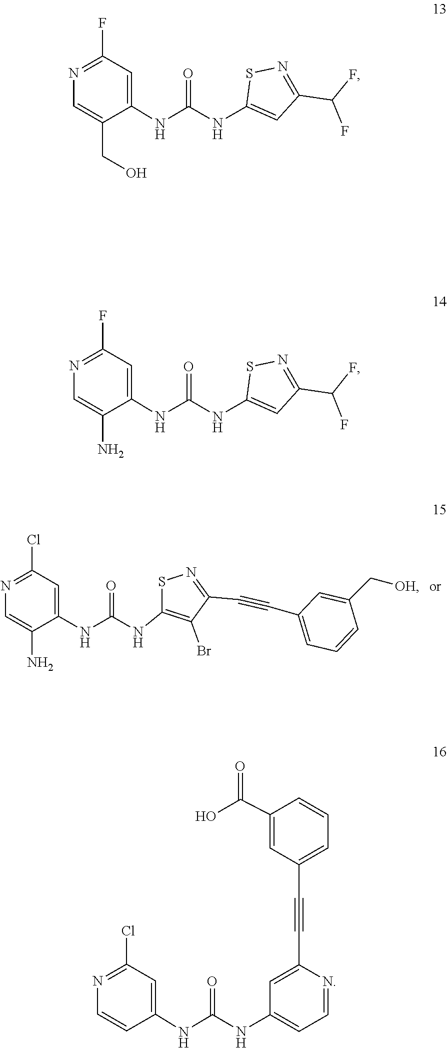 [SMILES: O=C(Nc1cc(C(F)F)ns1)Nc1cc(F)ncc1CO ; Nc1cnc(F)cc1NC(=O)Nc1cc(C(F)F)ns1 ; Nc1cnc(Cl)cc1NC(=O)Nc1snc(C#Cc2cccc(CO)c2)c1Br ; O=C(Nc1ccnc(Cl)c1)Nc1ccnc(C#Cc2cccc(C(=O)O)c2)c1]

In some embodiments, the small molecule compound, or a pharmaceutically acceptable salt thereof is a degrader. In some embodiments, the degrader has the structure of Formula IV:

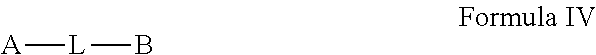

wherein A is a BRG1 and/or BRM binding moiety; L is a linker; and B is a degradation moiety, or a pharmaceutically acceptable salt thereof. In some embodiments, the degradation moiety is a ubiquitin ligase moiety. In some embodiments, the ubiquitin ligase binding moiety includes Cereblon ligands, IAP (Inhibitors of Apoptosis) ligands, mouse double minute 2 homolog (MDM2), hydrophobic tag, or von Hippel-Lindau ligands, or derivatives or analogs thereof.

In some embodiments, A is a BRG1 binding moiety. In some embodiments, A is a BRM binding moiety. In some embodiments, A includes the structure of any one of Formula 1-Ill, or any one of compounds 1-16.

In some embodiments, the hydrophobic tag includes a diphenylmethane, adamantine, or tri-Boc arginine, i.e., the hydrophobic tag includes the structure:

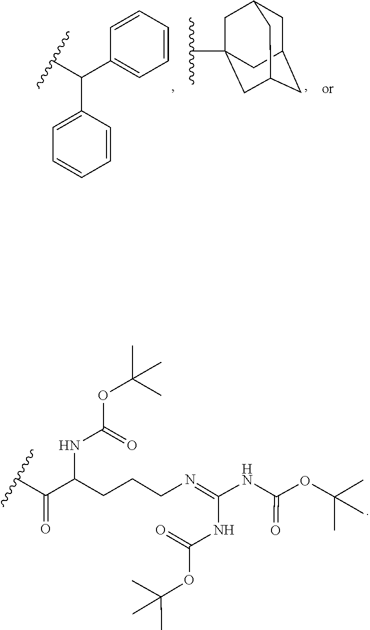

In some embodiments, the ubiquitin ligase binding moiety includes the structure of Formula A:

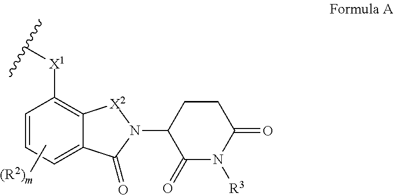

wherein X¹ is CH₂, O, S, or NR¹, wherein R¹ is H, optionally substituted C₁-C₆ alkyl, or optionally substituted C₁-C₆ heteroalkyl; X² is C═O, CH₂, or

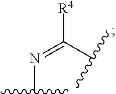

R³ and R⁴ are, independently, H, optionally substituted C₁-C₆ alkyl, or optionally substituted C₁-C₆ heteroalkyl; m is 0, 1, 2, 3, or 4; and each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,
or a pharmaceutically acceptable salt thereof.

In some embodiments, the ubiquitin ligase binding moiety includes the structure:

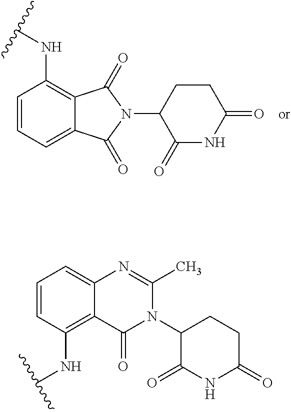 [SMILES: *Nc1cccc2c1C(=O)N(C1CCC(=O)NC1=O)C2=O ; *Nc1cccc2nc(C)n(C3CCC(=O)NC3=O)c(=O)c12]

or is a derivative or an analog thereof, or a pharmaceutically acceptable salt thereof.

In some embodiments, the ubiquitin ligase binding moiety includes the structure of Formula B:

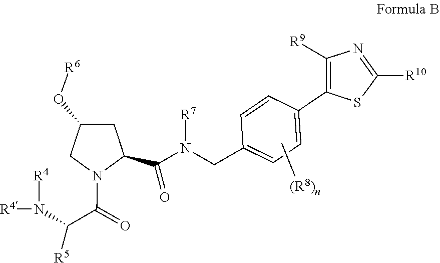

wherein each R⁴, R^{4′}, and R⁷ is, independently, H, optionally substituted C₁-C₆ alkyl, or optionally substituted C₁-C₆ heteroalkyl; R⁵ is optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R⁶ is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; n is 0, 1, 2, 3, or 4; each R⁸ is, independently, halogen, optionally substituted C₁—C alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and each R⁹ and R¹⁰ is, independently, H, halogen, optionally substituted C₁-C₆ alkyl, or optionally substituted C₆-C₁₀ aryl, wherein R^{4′} or R⁵ includes a bond to the linker, or a pharmaceutically acceptable salt thereof.

In some embodiments, the ubiquitin ligase binding moiety includes the structure:

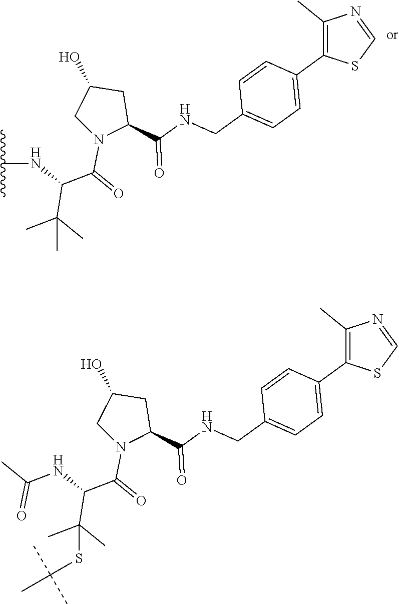

or is a derivative or analog thereof, or a pharmaceutically acceptable salt thereof.

In some embodiments, the ubiquitin ligase binding moiety includes the structure of Formula C:

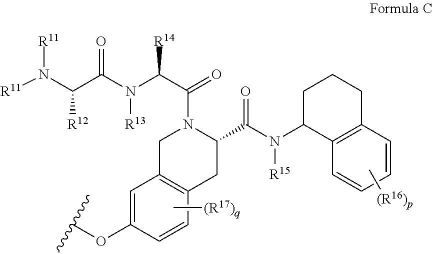

wherein each R¹¹, R¹³, and R¹⁵ is, independently, H, optionally substituted C₁-C₆ alkyl, or optionally substituted C₁-C₆ heteroalkyl; R¹² is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R¹⁴ is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; p is 0, 1, 2, 3, or 4; each R¹⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; q is 0, 1, 2, 3, or 4; and each R¹⁷ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

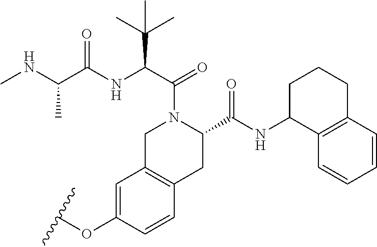 [SMILES: *Oc1ccc2c(c1)CN(C(=O)[C@@H](NC(=O)[C@H](C)NC)C(C)(C)C)[C@H](C(=O)NC1CCCc3ccccc31)C2]

or is a derivative or an analog thereof, or a pharmaceutically acceptable salt thereof.

In some embodiments, the ubiquitin ligase binding moiety includes the structure of Formula D:

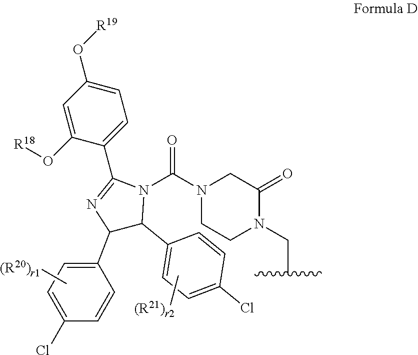

wherein each R¹⁸ and R¹⁹ is, independently, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; r1 is 0, 1, 2, 3, or 4; each R²⁰ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; r2 is 0, 1, 2, 3, or 4; and each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

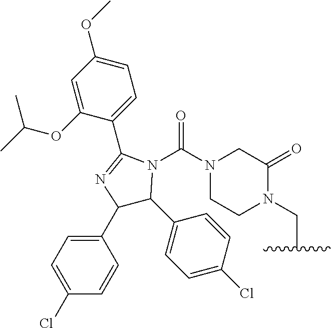 [SMILES: *CN1CCN(C(=O)N2C(c3ccc(OC)cc3OC(C)C)=NC(c3ccc(Cl)cc3)C2c2ccc(Cl)cc2)CC1=O]

or is a derivative or an analog thereof, or a pharmaceutically acceptable salt thereof.

In some embodiments, the linker has the structure of Formula V:

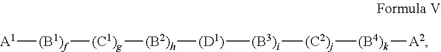

wherein A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)1-(B⁴)ₖ-A².

In some embodiments, D is optionally substituted C₂-C₁₀ polyethylene glycol. In some embodiments, C¹ and C² are each, independently, a carbonyl or sulfonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen or optionally substituted C₁₋₄ alkyl. In some embodiments, B¹, B², B³, and B⁴ each, independently, is selected from optionally substituted C₁-C₂ alkyl or optionally substituted C₁-C₃ heteroalkyl. In some embodiments, j is 0. In some embodiments, k is 0. In some embodiments, j and k are each, independently, 0. In some embodiments, f, g, h, and i are each, independently, 1.

In some embodiments, the linker of Formula V has the structure of Formula Va:

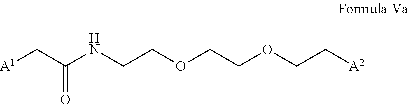

wherein A¹ is a bond between the linker and A, and A² is a bond between B and the linker.

In some embodiments, D is optionally substituted C₁₋₁₀ alkyl. In some embodiments, C₁ and C₂ are each, independently, a carbonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}, wherein R^{N} is hydrogen or optionally substituted C₁₋₄ alkyl. In some embodiments, B¹, B², B³, and B⁴ each, independently, is selected from optionally substituted C₁-C₂ alkyl, O, S, S(O)₂, and NR^{N}, wherein R^{N} is hydrogen or optionally substituted C₁₋₄ alkyl. In some embodiments, B¹ and B⁴ each, independently, is optionally substituted C₁-C₂ alkyl. In some embodiments, B¹ and B⁴ each, independently, is C₁ alkyl. In some embodiments, B² and B⁴ each, independently, is NR^{N}, wherein R^{N} is hydrogen or optionally substituted C₁₋₄ alkyl. In some embodiments, B² and B⁴ each, independently, is NH. In some embodiments, f, g, h, l, j, and k are each, independently, 1.

In some embodiments, the linker of Formula V has the structure of Formula Vb:

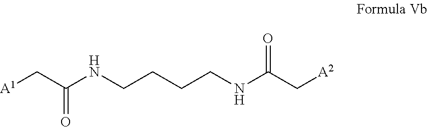

wherein A¹ is a bond between the linker and A, and A² is a bond between B and the linker.

Preferably, the compound used in the methods disclosed herein is of the following structure:

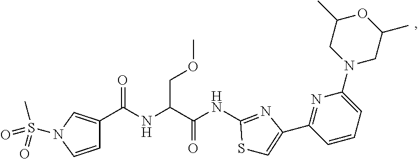 [SMILES: COCC(NC(=O)c1ccn(S(C)(=O)=O)c1)C(=O)Nc1nc(-c2cccc(N3CC(C)OC(C)C3)n2)cs1]

or a pharmaceutically acceptable salt thereof.

More preferably, the compound used in the methods disclosed herein is of the following structure:

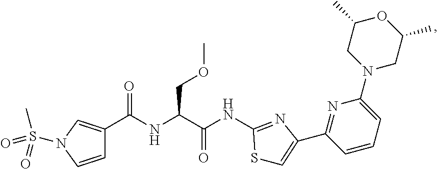 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

or a pharmaceutically acceptable salt thereof.

### Combination Formulations and Uses Thereof

The compounds of the disclosure can be combined with one or more therapeutic agents. In particular, the therapeutic agent can be one that treats or prophylactically treats cancer.

### Combination Therapies

An agent of the disclosure can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds, when combined, should provide a therapeutic effect.

Reduction in the metastatic potential of a cancer allows the agent of the disclosure to be used as an adjuvant, e.g., after or in conjunction with surgical intervention, radiation therapy, or chemotherapy. Accordingly, in some embodiments, the method further includes surgical intervention, radiation therapy, or chemotherapy to treat the cancer in the subject (e.g., the administering step is performed after or in conjunction with the surgical intervention, radiation therapy, or chemotherapy).

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. Also included is 5-fluorouracil (5-FU), leucovorin (LV), irinotecan, oxaliplatin, capecitabine, paclitaxel and docetaxel. Non-limiting examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylolmelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma and calicheamicin omega (see, e.g., Agnew, Chem. Intl. Ed Engl. 33:183-186 (1994)); dynemicin, including dynemicin A; bisphosphonates, such as clodronate; an esperamicin (as well as neocarzinostatin chromophore and related chromoprotein enediyne antibiotic chromophores), aclacinomycin, actinomycin, authramycin, azaserine, bleomycin, cactinomycin, carubicin, carzinophilin, chromomycin, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and anatoxins; mitoguazone; mitoxantrone; mopidamol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verrucarin A, roridin A, and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, III.), and Taxotere® docetaxel (Rhone-Poulenc Rorer, Antony, France); chlorambucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinnorelbine; novantrone; teniposide; edatrexate; daunomycin; aminopterin; Xeloda®; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan® (Rituximab); Zenapax (Daclizumab); Simulect (Basiliximab); Synagis (Palivizumab); Remicade® (Infliximab); Herceptin (Trastuzumab); Mylotarg (Gemtuzumab ozogamicin); Campath (Alemtuzumab); Zevalin® (Ibritumomab tiuxetan); Humira® (Adalimumab); Xolair® (Omalizumab); Bexxar (Tositumomab-1-131); Raptiva® (Efalizumab); Erbitux (Cetuximab); Avastin® (Bevacizumab); Tysabri® (Natalizumab); Actemra® (Tocilizumab); Vectibix® (Panitumumab); Lucentis® (Ranibizumab); Soliris® (Eculizumab); Cimzia® (Certolizumab pegol); Simponi® (Golimumab); Ilaris® (Canakinumab); Stelara® (Ustekinumab); Arzerra® (Ofatumumab); Prolia® (Denosumab); Numax (Motavizumab); ABThrax (Raxibacumab); Benlysta (Belimumab); Yervoy® (Ipilimumab); Adcetris® (Brentuximab Vedotin); Perjeta® (Pertuzumab); Kadcyla® (Ado-trastuzumab emtansine); and Gazyva® (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, which interacts with a checkpoint protein. In some embodiments, the inhibitor of checkpoint is an agent, such as an antibody, which interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK1, CHK2, A2aR, B-7 family ligands, or a combination thereof.

In some embodiments, the compound of the disclosure is used in combination with another anti-cancer therapy used for the treatment of uveal melanoma such as surgery, a MEK inhibitor, and/or a PKC inhibitor, or a combination thereof. For example, in some embodiments, the method further comprises performing surgery prior to, subsequent to, or at the same time as administration of the compound of the disclosure. In some embodiments, the method further comprises administration of a MEK inhibitor (e.g., selumetinib, binimetinib, or tametinib) and/or a PKC inhibitor (e.g., sotrastaurin or IDE196) prior to, subsequent to, or at the same time as administration of the compound of the disclosure.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In some embodiments, the compound of the disclosure is used in combination with another anti-cancer therapy used for the treatment of uveal melanoma such as surgery, a MEK inhibitor, and/or a PKC inhibitor, or combinations thereof. For example, in some embodiments, the method further comprises performing surgery prior to, subsequent to, or at the same time as administration of the compound of the invention. In some embodiments, the method further comprises administration of a MEK inhibitor (e.g., selumetinib, binimetinib, or tametinib) and/or a PKC inhibitor (e.g., sotrastaurin or IDE196) prior to, subsequent to, or at the same time as administration of the compound of the disclosure.

In some embodiments, the anticancer therapy and the compound of the disclosure are administered within 28 days (e.g., within 21 days, within 14 days, or within 7 days) of each other and each in an amount that, when combined, is effective to treat the subject.

### Pharmaceutical Compositions

The compounds of the present disclosure may be formulated into pharmaceutical compositions for administration to human subjects in a biologically compatible form suitable for administration in vivo. Pharmaceutical compositions typically include an active agent as described herein and a physiologically acceptable excipient (e.g., a pharmaceutically acceptable excipient). Formulation principles for the compounds disclosed herein may be those described, e.g., in WO 2020/160180, the disclosure of which is incorporated by reference herein in its entirety.

The compounds ofthe disclosure may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. Preferably, the compound is administered orally.

Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary).

The compound described herein may be formulated into a unit dosage form for oral administration (e.g., a capsule) as described in Table 1. The API in Table 1 is a compound of the following structure:

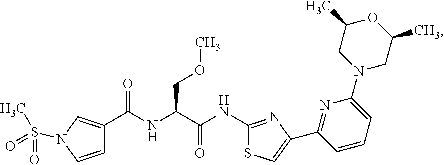 [SMILES: COC[C@H](NC(=O)c1ccn(S(C)(=O)=O)c1)C(=O)Nc1nc(-c2cccc(N3C[C@@H](C)O[C@@H](C)C3)n2)cs1]

TABLE 1

| | | 2.5 mg Capsule | | 20 mg Capsule | |
|---|---|---|---|---|---|
| | | Amount | | Amount | |
| | | per | | per | |
| | | Capsule | Content | Capsule | Content |
| Component | Function | (mg) | (%) | (mg) | (%) |
| API | Active | 2.50 | 2.63 | 20.00 | 17.39 |
| | ingredient | | | | |
| Microcrystalline | Filler | 48.24 | 50.78 | 48.20 | 41.91 |
| cellulose | | | | | |
| Mannitol | Filler | 36.18 | 38.08 | 36.95 | 32.13 |
| Croscarmellose | Disin- | 4.75 | 5.00 | 5.80 | 5.04 |
| Sodium | tegrant | | | | |
| Sodium lauryl | Wetting | 0.95 | 1.00 | 1.15 | 1.00 |
| sulfate | agent | | | | |
| Colloidal | Glidant | 1.90 | 2.00 | 2.30 | 2.00 |
| silicon dioxide | | | | | |
| Magnesium | Lubricant | 0.48 | 0.51 | 0.60 | 0.52 |
| stearate | | | | | |
| Size 4, Swedish | Capsule | 1 capsule | — | — | — |
| Orange HPMC | Shell | | | | |
| Capsules | | | | | |
| Size 3, Blue | Capsule | — | — | 1 capsule | — |
| Green HPMC | Shell | | | | |
| Capsules | | | | | |
| Total | | 95.00 | 100 | 115.00 | 100 |

The composition of the Swedish orange hypromellose capsule shells is described in Table 2.

TABLE 2

| Composition of | | |
|---|---|---|
| capsule body and cap | Function | Content |
| FDA/E172 RED IRON | Colorant | 1.1817% |
| OXIDE | | |
| TITANIUM DIOXIDE | Opacifier | 0.4916% |
| Hypromellose | Structure | qs 100% |

The composition of blue green hypromellose capsule shells is described in Table 3.

TABLE 3

| Composition of | | |
|---|---|---|
| capsule body and cap | Function | Content |
| FD&C Blue #1 | Colorant | 0.0281% |
| FD&C Yellow #5 | Colorant | 0.0069% |
| TITANIUM DIOXIDE | Opacifier | 2.2306% |
| Hypromellose | Structure | qs 100% |

### EXAMPLES

### Example 1. Compound a has a Positive Impact on Clinically Relevant Markers Associated with Poor Prognosis of Uveal Melanoma

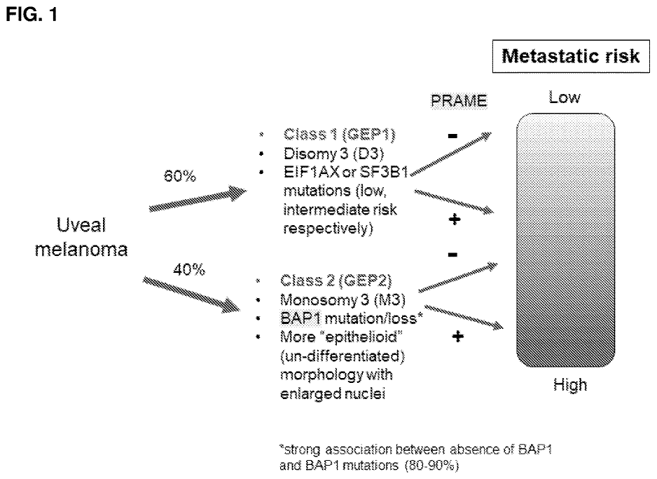

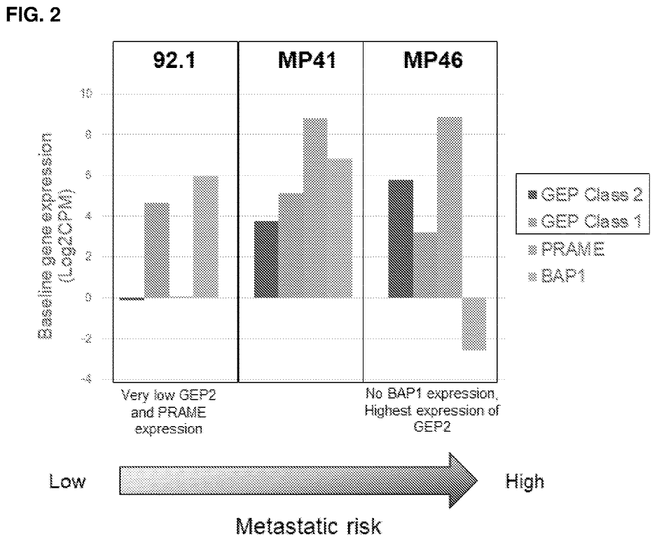

Uveal melanoma can be classified into Class 1 or Class 2 tumors based on the DecisionDx®-UM gene expression profiling (GEP) test, a widely used prognostic test to predict individual risk of metastasis in patients with uveal melanoma. Class 1 and 2 tumors are associated with low and high risk of metastasis, respectively. Gene expression profiling (GEP) Class 2 (dark blue), GEP Class 1 (light blue), PRAME (green), and BAP1 (orange) expression were measured in three uveal melanoma (UM) CDX models: 92-1, MP-41, and MP-46 (FIG. 2). GEP class 1 and GEP class 2 are defined in FIG. 1.

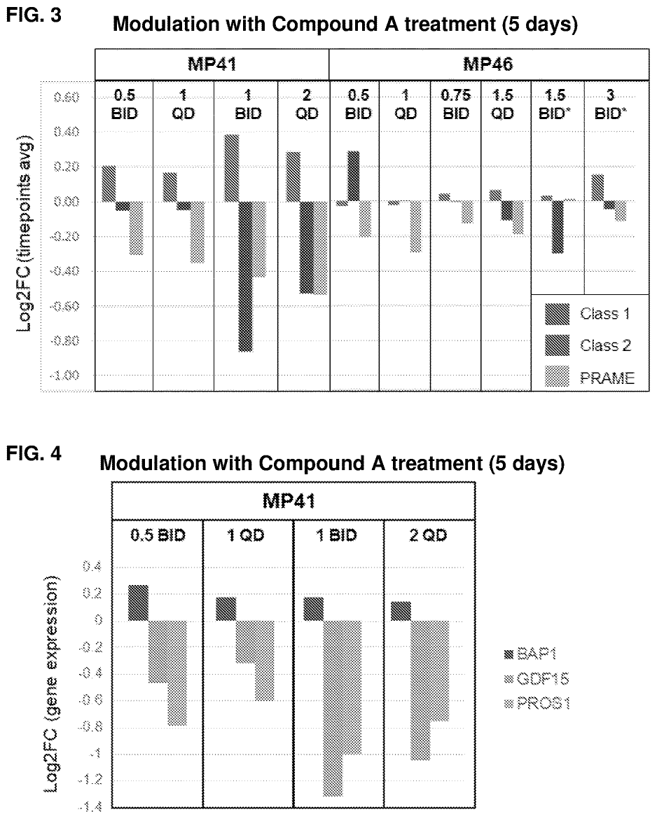

Differential gene expression was measured by RNA-seq in two uveal melanoma (UM) CDX models: MP-41 and MP-46 (FIG. 3). UM CDX model MP-41 was divided into four cohorts—cohort one was treated with 0.5 mpk of Compound A twice a day (BID), cohort two was treated with 1 mpk of Compound A once a day (QD), cohort three was treated with 1 mpk of Compound A twice a day, and cohort 4 was treated with 2 mpk of Compound A once a day. UM CDX model MP-46 was divided into six cohorts —cohort one was treated with 0.5 mpk of Compound A twice a day (BID), cohort two was treated with 1 mpk of Compound A once a day (QD), cohort three was treated with 0.75 mpk of Compound A twice a day, cohort 4 was treated with 1.5 mpk of Compound A once a day, cohort five was treated with 1.5 mpk of Compound A twice a day, and cohort six was treated with 3 mpk of Compound A twice a day. All treatments ended at 5 days, and animals were sacrificed. GEP Class 1 (green) comprises 8 genes that are high in Class 1 (low risk), and GEP Class 2 (blue) comprises 4 genes that are high in Class 2 (higher risk). PRAME (grey) positivity is associated with poor prognosis in several malignancies, including UM.

Treatment with Compound A results in a decrease in PRAME and a positive shift in the DecisionDx®-UM Gene Expression Profile (GEP), with a decrease in higher risk profile Class 2 genes.

### Example 2. BRG1/BRM Inhibition May Lead to a Less Suppressive Tumor Microenvironment in Uveal Melanoma

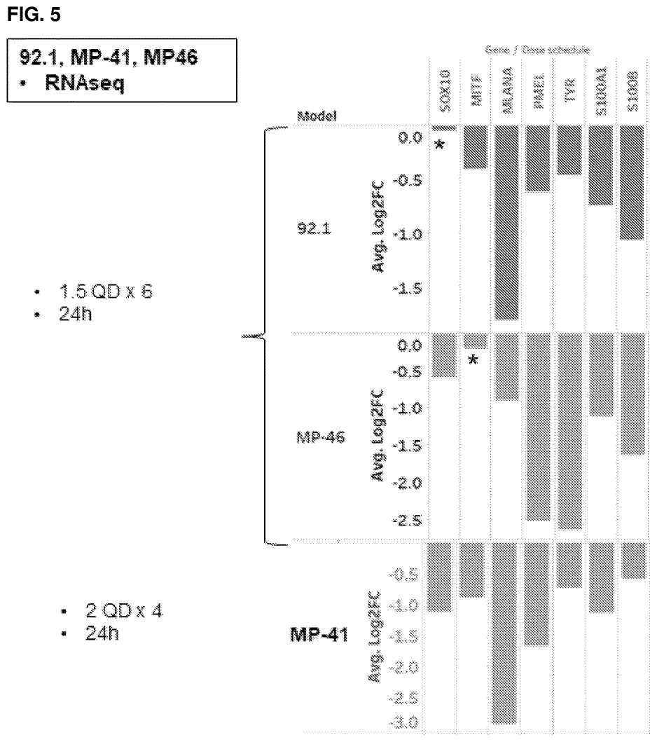

Differential gene expression was measured by RNA-seq in uveal melanoma (UM) CDX model: MP-41 (FIG. 4). This CDX model is a BAP1 intact but aggressive uveal melanoma model. UM CDX model MP-41 was divided into four cohorts—cohort one was treated with 0.5 mpk of Compound A twice a day (BID), cohort two was treated with 1 mpk of Compound A once a day (QD), cohort three was treated with 1 mpk of Compound A twice a day, and cohort 4 was treated with 2 mpk of Compound A once a day. All treatments ended at 4 or 5 days, and animals were sacrificed.

Treatment with Compound A leads to subtle upregulation of BAP1 (grey) and dose-dependent downregulation of two genes found to be upregulated with BAP1 loss: GDF15 (green) and PROS1 (blue). PROS1 has been mechanistically linked to immunosuppressive macrophage polarization in UM in preclinical studies.

### Example 3. BRG1/BRM Inhibition Downregulates the MITF/SOX10 Pathway

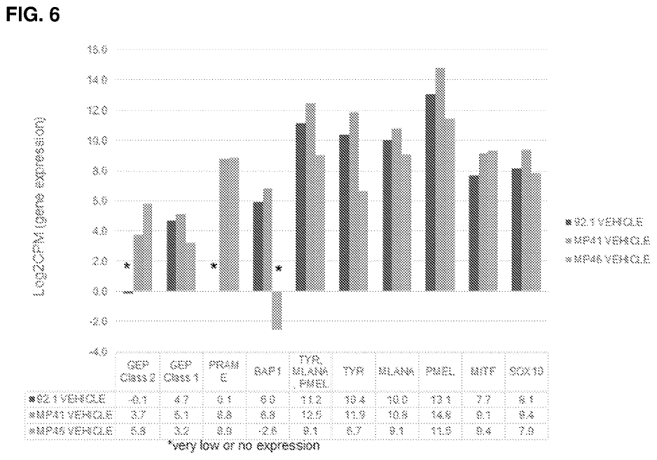

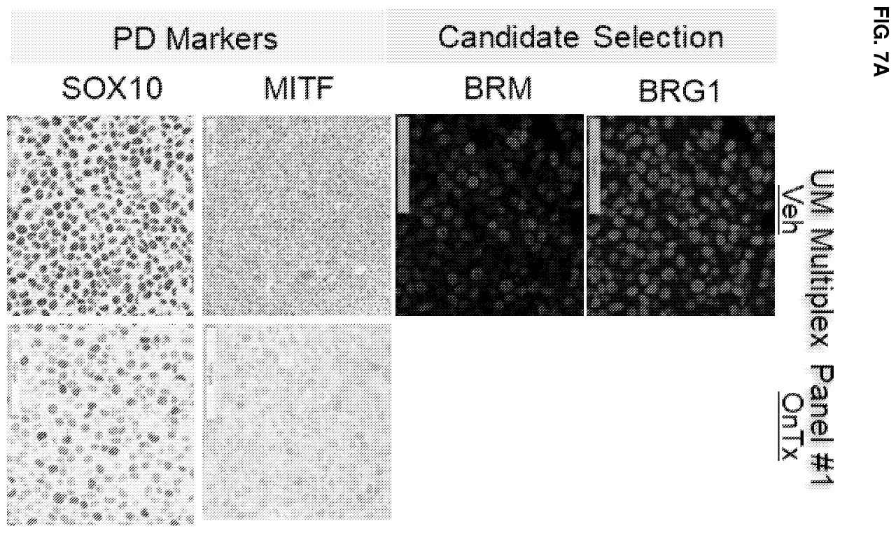

Differential gene expression was measured by RNA-seq in three uveal melanoma (UM) CDX models: 92-1, MP-41, and MP-46 (FIG. 5 and FIG. 6). UM CDX models 92.1 and MP-46 were treated with 1.5 mpk of Compound A, daily (QD), for 6 days, and then animals were sacrificed (FIG. 5). UM CDX model MP-41 was treated with 2 mpk of Compound A, daily, for 4 days, and then animals were sacrificed (FIG. 5). Treatment with a vehicle is shown in FIG. 6. Expression of SOX10, MITF (PD markers) and their downstream targets: melanin pathway markers MLANA, PMEL, and TYR were analyzed. Cyclin D1 is a cell proliferation marker, and BRM and BRG1 were analyzed for candidate selection.

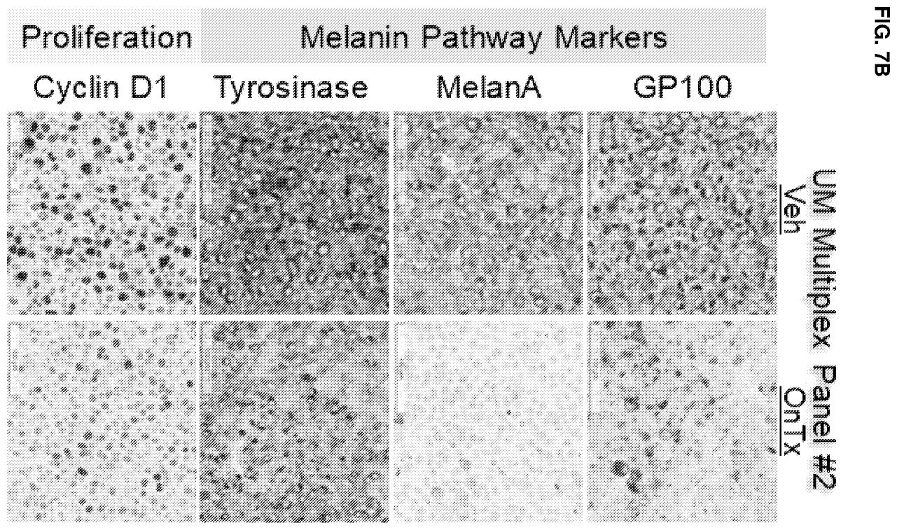

Representative MP-41 CDX tumor slices were analyzed after 5 days of treatment with 2 mpk of Compound A (OnTx) or a Vehicle (Veh), administered daily (FIGS. 7A and 7B). Representative immunohistochemistry (IHC) images show a correlation with gene expression changes and shows intratumor heterogeneity. Proliferation marker, Cyclin D1, decreases with treatment.

Target engagement markers were well-expressed across preclinical models (FIG. 6). Compound A treatment leads to sustained downregulation of lineage transcription factors, MITF and SOX10, and their melanoma-specific downstream targets across multiple uveal melanoma models (FIG. 5). Decrease in proliferation markers associated with impact of treatment on tumor growth and melanin pathway markers associated with uveal melanoma diagnosis located downstream of MITF/SOX10 signaling was demonstrated in response to FHD-286 treatment in MP-41 xenograft studies (FIGS. 7A and 7B).

### OTHER EMBODIMENTS

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

What is claimed is:

1. A method of slowing the spread of a migrating cancer in a subject having a high risk of the spread of the migrating cancer, the method comprising administering to the subject an effective amount of an agent that reduces the level and/or activity of BRG1 and/or BRM.

2. A method of reducing the rate of tumor seeding of a cancer in a subject having a high risk of tumor seeding of a cancer, the method comprising administering to the subject an effective amount of an agent that reduces the level and/or activity of BRG1 and/or BRM.

3. A method of reducing or treating metastatic nodule-forming of a cancer in a subject having a high risk of metastatic nodule-forming of a cancer, the method comprising administering to the subject an effective amount of an agent that reduces the level and/or activity of BRG1 and/or BRM.

4. A method of reducing metastatic risk of a cancer in a subject having a high metastatic risk, the method comprising administering to the subject an effective amount of an agent that reduces the level and/or activity of BRG1 and/or BRM.

5. A method for treating metastatic cancer in a subject having a high metastatic risk, the method comprising administering to the subject an effective amount of an agent that reduces the level and/or activity of BRG1 and/or BRM.

6. A method for inhibiting cancer cell invasion or migration in a subject having a high risk of cancer cell invasion or migration, the method comprising administering to the subject an effective amount of an agent that reduces the level and/or activity of BRG1 and/or BRM.

7. The method of any one of claims 1 to 6, wherein the cancer is melanoma, non-small cell lung cancer, small-cell lung cancer, ovarian cancer, breast cancer, esophagogastric cancer, oral cancer, glioblastoma, colon cancer, hematologic cancer, sarcoma, or neuroblastoma.

8. The method of claim 7, wherein the cancer is melanoma.

9. The method of claim 8, wherein the melanoma is uveal melanoma, mucosal melanoma, or cutaneous melanoma.

10. The method of claim 9, wherein the melanoma is uveal melanoma.

11. The method of claim 7, wherein the cancer is a hematologic cancer.

12. The method of claim 11, wherein the hematologic cancer is acute myeloid leukemia.

13. The method of claim 12, wherein the acute myeloid leukemia is refractory acute myeloid leukemia.

14. The method of claim 12, wherein the acute myeloid leukemia is relapsed acute myeloid leukemia.

15. The method of claim 7, wherein the cancer is breast cancer.

16. The method of claim 15, wherein the breast cancer is an ER positive breast cancer, an ER negative breast cancer, triple positive breast cancer, or triple negative breast cancer.

17. The method of claim 15, wherein the breast cancer is a triple negative breast cancer.

18. The method of claim 7, wherein the cancer is an oral cancer that is an oral squamous cancer.

19. A method of treating GEP2 uveal melanoma in a subject in need thereof, and the method comprising administering to the subject an agent that reduces the level and/or activity of BRG1 and/or BRM.

20. A method of transforming a GEP2 uveal melanoma to a GEP1 uveal melanoma in a subject in need thereof, the method comprising administering to the subject an agent that reduces the level and/or activity of BRG1 and/or BRM.

21. The method of any one of claims 1 to 20, wherein the cancer expresses BRG1 and/or BRM protein.

22. The method of any one of claims 1 to 21, wherein the cancer expresses PRAME.

23. The method of any one of claims 1 to 22, wherein the cancer expresses PROS1.

24. The method of any one of claims 1 to 23, wherein the subject or cancer underexpresses BAP1, MGP, CXCL14, CLCA2, S100A8, BTG1, SAP130, ARG1, KRT6B, GJA, ID2, EIF1B, S100A9, CRABP2, KRT14, ROBO1, RBM23, TACSTD2, DSC1, SPRR1B, TRIM29, AQP3, TYRP1, PPL, LTA4H, or CST6.

25. The method of any one of claims 1 to 24, wherein the subject or cancer overexpresses SPP1.

26. The method of any one of claims 1 to 25, wherein the subject expresses PRAME.

27. The method of any one of claims 1 to 26, wherein the subject expresses PROS1.

28. The method of any one of claims 1 to 27, wherein the subject overexpresses SNA11, TCF4, TWIST1, FOXC2, IL1RN, MMP2, SOX10, WNT11, MMP3, PDGFRB, or JAG1.

29. A method of reducing the expression of PRAME and/or PROS1 in a subject in need thereof, the subject having a high metastatic risk and expressing PRAME and/or PROS1, the method comprising administering to the subject an effective amount of an agent that reduces the level and/or activity of BRG1 and/or BRM.

30. The method of any one of claims 1 to 29, wherein the method further comprises surgical intervention, radiation therapy, or chemotherapy to treat the cancer in the subject.

31. The method of claim 30, wherein the administering step is performed after or in conjunction with the surgical intervention, radiation therapy, or chemotherapy.

32. The method of any one of claims 1 to 31, wherein the administering step is performed as an adjuvant therapy.

33. The method of any one of claims 1 to 32, wherein the agent is administered at a total dose of 2.5-10 mg daily or 2.5-10 mg every other day.

34. The method of any one of claims 1 to 32, wherein the agent is administered in a total dose of 2.5-7.5 mg daily or 2.5-7.5 mg every other day.

35. The method of any one of claims 1 to 32, wherein the agent is administered in a total dose of 2.5-5.0 mg daily or 2.5-5.0 mg every other day.

36. The method of any one of claims 1 to 32, wherein the agent is administered in a total dose of 5.0-7.5 mg daily or 5.0-7.5 mg every other day.

37. The method of any one of claims 1 to 32, wherein the agent is administered in a total dose of 2.5 mg daily or 2.5 mg every other day.

38. The method of any one of claims 1 to 32, wherein the agent is administered in a total dose of 5.0 mg daily or 5.0 mg every other day.

39. The method of any one of claims 1 to 32, wherein the agent is administered in a total dose of 7.5 mg daily or 7.5 mg every other day.

40. The method of any one of claims 1 to 32, wherein the agent is administered in a total dose of 10 mg daily or 10 mg every other day.

41. The method of any one of claims 1 to 40, wherein the agent is administered to the subject once daily.

42. The method of any one of claims 1 to 40, wherein the compound or pharmaceutically acceptable salt thereof is administered to the subject once every other day.

43. The method of any one of claims 1 to 42, wherein the agent is administered orally.

44. The method of claim 43, wherein the agent is administered in a unit dosage form selected from a capsule or a tablet.

45. The method of any one of claims 1 to 44, wherein, if the subject experiences dose limiting toxicity following administration of the agent, the method further comprises interrupting administration of the agent.

46. The method of claim 45, wherein the administration of the agent is interrupted for a duration sufficient for the dose limiting toxicity to resolve.

47. The method of claim 46, wherein, after the dose limiting toxicity is resolved, the method further comprises resuming administration of the agent.

48. The method of claim any one of claims 45 to 47, wherein the dose limiting toxicity is characterized by keratitis.

49. The method of claim 48, wherein the dose limiting toxicity is characterized by Grade 3 keratitis.

50. The method of any one of claims 45 to 49, wherein the dose limiting toxicity is characterized by hyperbilirubinemia.

51. The method of claim 50, wherein the dose limiting toxicity is characterized by Grade 3 hyperbilirubinemia.

52. The method of any one of claims 1 to 51, wherein the subject is a human.

53. The method of any one of claims 1 to 52, wherein the method decreases metastasis to the liver and brain.

54. The method of any one of claims 1 to 53, wherein the method reduces cancer tumor growth in the subject compared to a subject that is not administered the agent.

55. The method of any one of claims 1 to 54, wherein the method suppresses metastatic progression of cancer in the subject compared to a subject that is not administered the agent.

56. The method of any one of claims 1 to 55, wherein the method suppresses metastatic colonization of cancer in the subject compared to a subject that is not administered the agent.

57. A method for inhibiting proliferation or growth of cancer stem cells or cancer initiating cells, comprising contacting the cell with an agent that reduces the level and/or activity of BRG1 and/or BRM in an amount sufficient to inhibit proliferation or growth of the cell.

58. The method of any one of claims 1 to 57, wherein the agent is a small molecule inhibitor of BRG1 and/or BRM.

59. The method of any one of claims 1 to 58, wherein the agent inhibits BRG1.

60. The method of any one of claims 1 to 59, wherein the agent inhibits epithelial to mesenchymal transition.

61. The method of any one of claims 1 to 60, wherein the agent is a compound, N-(1-((4-(6-(2,6-dimethylmorpholino)pyridin-2-yl)thiazol-2-yl)amino)-3-methoxy-1-oxopropan-2-yl)-1-(methylsulfonyl)-1H-pyrrole-3-carboxamide, or a pharmaceutically acceptable salt thereof.

62. The method of any one of claims 1 to 60, wherein the compound is: 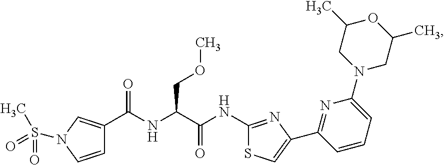 [SMILES: COC[C@H](NC(=O)c1ccn(S(C)(=O)=O)c1)C(=O)Nc1nc(-c2cccc(N3CC(C)OC(C)C3)n2)cs1]
or a pharmaceutically acceptable salt thereof.

63. The method of any one of claims 1 to 60, wherein the compound is: 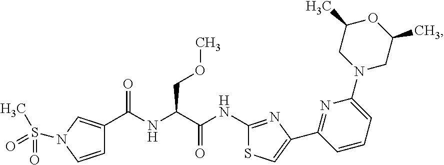 [SMILES: COC[C@H](NC(=O)c1ccn(S(C)(=O)=O)c1)C(=O)Nc1nc(-c2cccc(N3C[C@@H](C)O[C@@H](C)C3)n2)cs1]
or a pharmaceutically acceptable salt thereof.

64. The method of any one of claims 1 to 60, wherein the compound is: 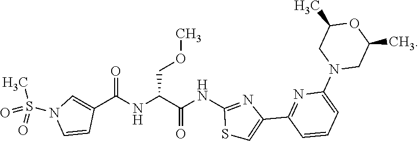 [SMILES: COC[C@@H](NC(=O)c1ccn(S(C)(=O)=O)c1)C(=O)Nc1nc(-c2cccc(N3C[C@@H](C)O[C@@H](C)C3)n2)cs1]
or a pharmaceutically acceptable salt thereof.
